(19) United States
(12) Patent Application Publication
(10) Pub. No.: US 2003/0171286 A1
(43) Pub. Date: Sep. 11, 2003

(54) Use and targeting of CD98 light-chain proteins in therapies for thyroid hormone disorders

(76) Inventors: Yun-Bo Shi, Gaithersburg, MD (US); Peter M. Taylor, Dundee (GB); George J. Peter, Fife (GB); James W.A. Ritchie, Aberdeenshire (GB)

Correspondence Address: KNOBBE MARTENS OLSON & BEAR LLP, 2040 MAIN STREET, FOURTEENTH FLOOR, IRVINE, CA, 92614, US

(21) Appl. No.: 10/307,063

(22) Filed: Nov. 27, 2002

Related U.S. Application Data
(63) Continuation of application No. PCT/US01/20843, filed on Jun. 28, 2001.
(60) Provisional application No. 60/215,414, filed on Jun. 30, 2000.

(51) Int. Cl. A61K 38/22; G01N 33/53; G01N 33/567; C07K 14/575

(52) U.S. Cl. 514/12; 530/399; 435/7.2

(57) ABSTRACT

The invention is related to the normalization of thyroid hormone transport by modulation of 4F2hc, CD98lc, and the 4F2hc-CD98lc heterodimer. Approaches to identify compounds that modulate thyroid hormone disorders, methods of identifying agonists and antagonists that modulate thyroid hormone disorders, and methods of making pharmaceuticals that ameliorate a thyroid hormone disorder are described herein.

### CROSS-REFERENCE TO RELATED APPLICATIONS

This application is a continuation of international application no. PCT/US01/20843, and claims the benefit of priority of international application no. PCT/US01/20843 having international filing date of Jun. 28, 2001, designating the United States of America and published in English, which claims the benefit of priority of U.S. provisional patent application No. 60/215,414, filed Jun. 30, 2000; both of which are hereby expressly incorporated by reference in their entireties.

### FIELD OF THE INVENTION

The invention is related to the normalization of thyroid hormone function by modulation of 4F2hc, CD98lc, and the 4F2hc-CD98lc heterodimer.

### BACKGROUND OF THE INVENTION

The thyroid gland produces both thyroxine (T₄) and tri-iodothyronine (T₃) and releases them into the blood circulation, although much circulating T₃ (the more active hormone) is generated by monodeiodination of T₄ in liver and kidney (Oppenheimer et al. 1996, in The Thyroid, pp 162-184, eds. L E Braverman & R Utiger, Philadelphia: Lippincott-Raven; Hennemann & Visser 1997, in Handbook of Experimental Pharmacology, v. 128, pp 75-117, eds. A P Weetman & A Grossman, Berlin/New York: Springer-Verlag). The major source of nuclear receptor-bound T₃ in many tissues (e.g. liver) is the blood T₃ pool, although some tissues (e.g. brain) generate T₃ endogenously from T₄ (Oppenheimer et al. 1996, in The Thyroid, pp 162-184, eds. L E Braverman & R Utiger, Philadelphia: Lippincott-Raven; Hennemann & Visser 1997, in Handbook of Experimental Pharmacology, v. 128, pp 75-117, eds. A P Weetman & A Grossman, Berlin/New York: Springer-Verlag). Movement of thyroid hormones between intra- and extra-cellular fluid compartments across the cell membrane is therefore an important step for modulation of hormone action and metabolism. Surprisingly, the specific mechanisms by which thyroid hormones cross the cell membrane are not fully understood, although movement by simple diffusion is likely to be a minor component of their total blood-tissue exchange (Hennemann et al. 1986, Endocrinol 119: 1870-1872; Blondeau et al. 1988, J Biol Chem 263: 2685-2692; Chantoux et al. 1995, J Neurochem 65: 2549-2554; Blondeau et al. 1993, J Neurochem 60: 1407-1413; Zhou et al 1992, Biochem J 281: 81-86). Thyroid hormone (TH) transport into cells is inhibited by a wide variety of substances, including certain amino acids (notably tryptophan) (Blondeau et al 1993, J Neurochem 60: 1407-1413; Zhou et al. 1990, J Biol Chem 265: 17000-17004; Samson et al. 1992, Biochim Biophys Acta 1108: 91-98; Kemp & Taylor 1997, Amer J Physiol 272: E809-E816), bilirubin (Chantoux et al. 1993, Mol Cel Endocrinol 97: 145-151), bilirubin conjugates (Chantoux et al. 1993, Mol Cel Endocrinol 97: 145-151) and various structurally-unrelated drugs (Chantoux et al. 1993, Mol Cel Endocrinol 97: 145-151; Abe et al. 1998, J Biol Chem 273: 22395-22401). Recent reports (Abe et al. 1998, J Biol Chem 273: 22395-22401; Friesema et al. 1999, Biochem Biophys Res Commun 254: 497-501) show that thyroid hormones and sulfated derivatives are transported by organic anion transporters such as Ntcp and oatp 1-3, but the molecular mechanism by which thyroid hormones and amino acids interact has not been elucidated. There is evidence for a close functional link between transport of aromatic amino acids and thyroid hormones in erythrocytes (Zhou et al. 1992, Biochem J 281: 81-86; Samson et al. 1992, Biochim Biophys Acta 1108: 91-98), hepatocytes (Blondeau et al. 1998, J Biol Chem 263: 2685-2692; Kemp & Taylor 1997, Amer J Physiol 272: E809-E816) (by System T in both cases), placental choriocarcinoma cells (Prasad et al 1994, Endocrinology 134: 574-581) and astrocytes (Blondeau et al. 1993, J Neurochem 60: 1407-1413) (by System L).

Recent studies have identified several members of a new family of amino acid permeases (e.g. LAT1, IU12, ASUR4 (Prasad et al. 1999, Biochem Biophys Res Commun 255: 283-288; Mastroberardino et al. 1998, Nature 395: 288-291; Torrents et al. 1998, J Biol Chem 262: 9574-9580) which exhibit activation of amino acid transport, having functional characteristics of System L, only when co-expressed with 4F2 heavy-chain (hc) glycoprotein. The highly-hydrophobic light-chain (lc) permeases interact covalently with 4F2hc to produce a functional, heteromeric “transporter unit” in the cell membrane (Prasad et al. 1999, Biochem Biophys Res Commun 255: 283-288; Mastroberardino et al. 1998, Nature 395: 288-291; Torrents et al. 1998, J Biol Chem 262: 9574-9580). The Xenopus laevis lc permease IU12 (Torrents et al. 1998, J Biol Chem 262: 9574-9580) is an early T₃-response gene up-regulated during intestinal development (Liang et al. 1997, Cell Res 7: 179-193) and it has been suggested that IU12 is involved in the signal transduction pathway of T₃-induced metamorphosis (Liang et al. 1997, Cell Res 7: 179-193).

### SUMMARY OF THE INVENTION

Thyroid hormone (TH) action and metabolism require hormone transport across cell membranes. We have investigated the possibility that THs are substrates of amino acid transport (System L) mediated by heterodimers of 4F2 heavy-chain (hc) and the light-chain (lc) permease IU12. Co-expression of 4F2hc and IU12 cDNAs injected into Xenopus oocytes induces saturable, Na⁺-independent transport of tri-iodothyronine (T₃), thyroxine (T₄), (Kₘ of 1.8 and 6.3 μM respectively), tryptophan and phenylalanine. Induced TH and tryptophan uptakes are inhibited by excess BCH (synthetic System L substrate). Induced TH uptake is also inhibited by excess reverse tri-iodothyronine (rT₃), but not by triodothyroacetic acid (TRIAC) (TH analogue lacking an amino acid moiety). T₃ and tryptophan exhibit reciprocal inhibition of their 4F2hc-IU12 induced uptake. Transport pathways produced by 4F2hc-lc permease complexes are therefore envisioned as important routes for movement and exchange of TH (as well as amino acids) across vertebrate cell membranes, with a role in modulating TH action.

### BRIEF DESCRIPTION OF THE DRAWINGS

FIG. 1. Membrane topology model of a heterodimeric amino acid-transporter. The label hc indicates the single transmembrane domain of the glycoprotein heavy-chain (4F2hc). Potential N-linked glycosylation sites are indicated by forks. The putative transmembrane domains of the lipophilic light-chain (IU12) are numbered 1-12. The putative cysteine residues involved in disulfide linkage are indicated.

FIG. 2. Uptake of thyroid hormones (T₃, T₄ at 0.1 μM) by Xenopus oocytes 4 days after nuclear injection of 4F2hc and IU12 cDNA alone or in combination. Control oocytes were injected with water. Each bar represents mean uptake±S.E.M. measured in 5 separate batches of oocytes (using 8-11 individual oocytes per batch). *, Uptake value significantly different from corresponding value in water-injected oocytes with p<0.01. Inset:—time courses of 0.1 μM [¹²⁵I]T₄ uptake into oocytes injected with 4F2IU12 DNAs or water (each point represents mean uptake±S.E.M. for 8-10 oocytes).

FIG. 3. (a) Uptake of T₃ by oocytes injected with 4F2hc-IU12 DNAs or water as a function of external T₃ concentration. Data are mean uptake±S.E.M. for 9-11 oocytes at each point, 4 days post-injection. Smallest error bars are masked by symbols. 4F2hc-IU12 induced T₃ transport had an apparent Kₘ of 1.8 μM and Vₘₐₓ of 6.4±0.3 pmol/oocyte.h. Tryptophan uptake in the same batch of oocytes had a Kₘ of 70 μM and Vₘₐₓ of 180±54 pmol/oocyte.h. (b) Concentration-dependent inhibition of 4F2hc-IU12 induced [³H]tryptophan uptake by unlabelled T₃ or tryptophan. Data show uptake of tryptophan (1 μM tracer) in presence of increasing concentrations of unlabelled inhibitor, as a percentage of control uptake in absence of inhibitor. Each point represents mean value±S.E.M. for 8-11 4F2hc-IU12-injected oocytes, after appropriate correction for uptake in water-injected oocytes.

FIG. 4. Inhibition of T₃ (0.1 μM) and tryptophan (1 μM) uptake by iodothyronines, tryptophan and 2-endoamino-bicycloheptane-2-carboxylic acid (BCH) in Xenopus oocytes injected with 4F2hc-IU12 cDNAs or water. Inhibitor concentrations were 10 μM for T₃ and T₄, 5 mM for BCH and 10 mM for tryptophan. Each bar represents uptake in presence of inhibitor as a percentage of control uptake measured in absence of inhibitor (mean value±S.E.M. for 7-11 oocytes). Control T₃ uptakes were 71±7 and 22±3 fmol/oocyte.h for 4F2hc-IU12- and water-injected oocytes respectively. Control tryptophan uptakes were 10.2±0.4 and 2.1±0.1 pmol/oocyte.h for 4F2hc-IU12- and water-injected oocytes respectively. Similar results were obtained using a different batch of oocytes. *, significant reduction of uptake in presence of inhibitor (p<0.005).

FIG. 5. Mean cytoplasmic and nuclear [¹²⁵I]T₃ uptake/binding±S.E.M. for single batch of 4F2hc-IU12-cDNA and water-injected oocytes (3-6 oocytes per data point). Oocytes were incubated in TMA-Cl⁻ uptake buffer containing 100 nM [¹²⁵I]T₃ for 2 hours. Values in parenthesis are fold increase in uptake/binding over water injected oocytes.

FIG. 6. Increasing T3 uptake into oocytes by overexpression of 4F2hc-IU12 results in a stimulation of thyroid-dependent gene transcription. Ratio of luminescent counts for each oocyte type compared to thyroid-responsive luciferase (TRE)-only injected oocytes. Data shown for n=4 batches (each 7-9 oocytes). Concentration of T₃ used was 60 nM. 4/I/R/T-4F2hc/IU12/RXRα/TRβ co-injected oocytes. RXRα/TRβ (heterodimers of RXR, or 9-cis retinoic acid receptor, and TR)—nuclear receptors for TH; *, significant change in luciferase activity compared to TRE-only injected oocytes (p<0.05). Inset: Preliminary data from one batch of oocytes showing the effect of 5 mM BCH on 10 nM T₃ stimulation of luciferase activity in 4/I/T (4F2/IU12/TRE) co-injected oocyte. All incubations were performed over a 24-hour time period.

FIG. 7. Effect of tryptophan (Trp) and BCH on T3-induced luciferase activity in BeWo cells. Values shown are mean luciferase activity (cpm/well)±S.E.M. for n=3 individual wells from a single plate. Similar results were obtained in a second experiment. The T3-luciferase reporter construct was prepared according to Menjo, M., et al., 1999, Thyroid, 9: 959-67, and was infected into BeWo cells using adenovirus (a generous gift of Dr Y. Hayashi, University of Chicago, USA). Infected cells were exposed to experimental conditions for 24 h prior to assay of luciferase activity.

### DETAILED DESCRIPTION OF THE INVENTION

The invention relates to a general principle that an amino acid transporter protein mediates thyroid hormone transport across vertebrate cell membranes. This protein is a member of the CD98 light-chain permease family that provides a previous missing link in the chain by which thyroid hormones in the blood reach the cell nucleus, where they are generally believed to exert their major biological effects. Thyroid hormone disorders are among the most common medical problems in the Western world and this discovery enables the design and development of different therapies for these disorders, based on the regulation of thyroid hormone entry into cells and thus its action.

The present invention has identified these CD98 light-chain proteins as thyroid hormone transporters and makes possible the targeting of these proteins using substances developed by rational drug design or otherwise. Such regulation provides a powerful way to control thyroid hormone disorders such as hypo- and hyper-thyroidism (including goiter). It addresses related problems including obesity. The application of techniques of gene therapy permits the tissue-specific introduction or up- or down-regulation of these transporters. Developmental abnormalities caused by excess or deficiency of thyroid hormones during pregnancy are also subject to be treated by various such types of therapies.

Here we provide evidence, which indicates that amino acid transport activity produced by 4F2hc-IU12 heterodimers accepts thyroid hormones (T4 and T3), as substrates. The 4F2hc-IU12 induced uptakes of T₃ and tryptophan in oocytes show mutual inhibition and are both Na⁺-independent and inhibited by excess BCH, confirming that the expressed transport activity is System L. To our knowledge, this is the first report of thyroid hormone transport by a cloned amino acid transporter. Other recent studies (Abe et al. 1998, J Biol Chem 273: 22395-22401; Friesama et al. 1999, Biochem Biophys Res Commun 254: 497-501) demonstrate that organic anion transporters also accept thyroid hormones (TH) and other iodothyronines as substrates, providing both Na⁺-dependent (Ntcp) (Friesema et al. 1999, Biochem Biophys Res Commun 254: 497-501) and Na⁺-independent (oatp 1-3) (Abe et al. 1998, J Biol Chem 273: 22395-22401; Friesama et al. 1999, Biochem Biophys Res Commun 254: 497-501) transport pathways. The present results indicate that amino acid transporters producing System L-like activity provide an important route for physiologically relevant movements of TH across cell membranes. Furthermore, in combination with the knowledge that TH are also substrates for organic anion transporters, the results provide a rational basis for explaining the wide variety of reported inhibitors of cellular TH transport (Zhou et al. 1990, J Biol Chem 265: 17000-17004; Samson et al. 1992, Biochim Biophys Acta 1108: 91-98; Kemp & Taylor 1997, Amer J Physiol 272: E809E816; Chantoux et al. 1993, Mol Cel Endocrinol 97: 145-151; Abe et al. 1998, J Biol Chem 273: 22395-22401).

Definitions

The term “isolated” requires that a material be removed from its original environment (e.g., the natural environment if it is naturally occurring). For example, a naturally occurring polynucleotide or polypeptide present in a living cell is not isolated, but the same polynucleotide or polypeptide, separated from some or all of the coexisting materials in the natural system, is isolated.

The term “purified” does not require absolute purity; rather it is intended as a relative definition, with reference to the purity of the material in its natural state. Purification of natural material to at least one order of magnitude, preferably two or three magnitudes, and more preferably four or five orders of magnitude is expressly contemplated.

The term “enriched” means that the concentration of the material is at least about 2, 5, 10, 100, or 1000 times its natural concentration (for example), advantageously 0.01% by weight. Enriched preparations of about 0.5%, 1%, 5%, 10%, and 20% by weight are also contemplated.

The 4F2 Heavy-Chain and CD98 Light-Chain Genes

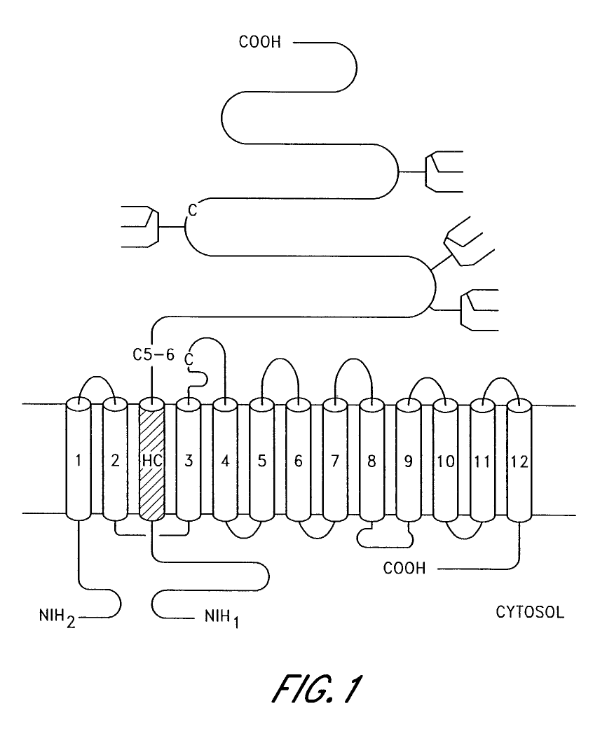

Amino acid transporter, 4F2 antigen (also known as CD98 antigen), is a heterodimeric protein composed of two subunits, a glycosylated heavy-chain that is approximately 80 kDa, and a non-glycosylated light-chain that is approximately 40 kDa (FIG. 1). 4F2hc/CD98hc (heavy-chain) is an integral membrane glycoprotein with an intracellular N terminus, a single membrane spanning domain, and a large extracellular domain with potential N-glycosylation sites. The gene 4F2hc/CD98hc is classified as gene SLC3A2. The GenBank Accession numbers for human membrane glycoprotein 4F2 antigen heavy chain mRNA are: J02939 (Nov. 8, 1994); M17430; M18811; J03569; NM002394 (Feb. 3, 2001), the sequences of which are all hereby expressly incorporated by reference in their entireties.

Xenopus laevis IU12 is a plasma membrane L-aminoacid transporter protein with 12 transmembrane domains and is a member of the family of CD98lc (light-chain) permease subunits which also include E16, LAT-1 and -2, and ASUR4, as well as others. IU12 is a homologue of human LAT1, which is classified as gene SLC7A5. The GenBank Accession Number for Xenopus laevis IU12 mRNA is AF019906 (Mar. 16, 1999), hereby expressly incorporated by reference in its entirety. The GenBank Accession Number for human E16 amino acid transporter mRNA is AF077866 (Sep. 26, 1998), hereby expressly incorporated by reference in its entirety. The GenBank Accession Number for human LAT-1 amino acid transporter mRNA is AF104032 (Mar 17, 1999), hereby expressly incorporated by reference in its entirety. The association of the light and heavy chain heterodimer is supposed to be linked by a disulfide bridge between extracellular cysteine residues on the respective heavy and light chains.

The 4F2hc nucleotide sequences of the invention include: (a) the cDNA sequences with GenBank Accession numbers: J02939; M17430; M18811; J03569; NM002394; (b) the nucleotide sequences that encode the amino acid sequences deduced from the cDNA sequences with GenBank Accession numbers: J02939; M17430; M18811; J03569; NM002394; (c) any nucleotide sequences that hybridize to the complement of the cDNA sequences given in GenBank Accession numbers: J02939; M17430; M18811; J03569; NM002394 under highly stringent conditions, e.g., hybridization to filter-bound DNA in 0.5 M NaHPO₄, 7% sodium dodecyl sulfate (SDS), 1 mM EDTA at 65 C., and washing in 0.1 times. SSC/0.1% SDS at 68 C. (Ausubel F. M. et al., eds., 1989, Current Protocols in Molecular Biology, Vol. I, Green Publishing Associates, Inc., and John Wiley & sons, Inc., New York, at p. 2.10.3) and encodes a functionally equivalent gene product; and (d) any nucleotide sequence that hybridizes to the complement of the cDNA sequence given in GenBank Accession numbers: J02939; M17430; M18811; J03569; NM002394 under less stringent conditions, such as moderately stringent conditions, e.g., washing in 0.2 times. SSC/0.1% SDS at 420 C. (Ausubel et al., 1989, supra), yet which still encodes a functionally equivalent gene product. Functional equivalents of 4F2hc include naturally occurring 4F2hcs present in other species, and mutant 4F2hcs, whether naturally occurring or engineered. The invention also includes degenerate variants of sequences (a) through (d).

The CD98lc nucleotide sequences of the invention include: (e) the cDNA sequence given in GenBank Accession Numbers AF019906; AF077866; AF104032; (f) the nucleotide sequences that encode the amino acid sequences deduced from the cDNA sequence given in GenBank Accession Numbers AF019906; AF077866; AF104032; (g) any nucleotide sequence that hybridizes to the complement of the cDNA sequence given in GenBank Accession Numbers AF019906; AF077866; AF104032 under highly stringent conditions, e.g., hybridization to filter-bound DNA in 0.5 M NaHPO₄, 7% sodium dodecyl sulfate (SDS), 1 mM EDTA at 65 C., and washing in 0.1 times. SSC/0.1% SDS at 68 C. (Ausubel F. M. et al., eds., 1989, Current Protocols in Molecular Biology, Vol. I, Green Publishing Associates, Inc., and John Wiley & sons, Inc., New York, at p. 2.10.3) and encodes a functionally equivalent gene product; and (h) any nucleotide sequence that hybridizes to the complement of the cDNA sequence given in GenBank Accession Numbers AF019906; AF077866; AF104032 under less stringent conditions, such as moderately stringent conditions, e.g., washing in 0.2 times. SSC/0.1% SDS at 42° C. (Ausubel et al., 1989, supra), yet which still encodes a functionally equivalent gene product. Functional equivalents of CD98lc include naturally occurring CD98lcs present in other species, and mutant CD98lcs, whether naturally occurring or engineered. The invention also includes degenerate variants of sequences (e) through (h).

The invention also includes nucleic acid molecules, preferably DNA molecules, that hybridize to, and are therefore the complements of, the nucleotide sequences (a) through (h), in the preceding paragraphs. Such hybridization conditions may be highly stringent or less highly stringent, as described above. In instances wherein the nucleic acid molecules are deoxyoligonucleotides (“oligos”), highly stringent conditions may refer, e.g., to washing in 6× SSC/0.05% sodium pyrophosphate at 37° C. (for 14-base oligos), 48° C. (for 17-base oligos), 55° C. (for 20-base oligos), and 60° C. (for 23-base oligos). These nucleic acid molecules may encode or act as 4F2hc or CD98lc antisense molecules, useful, for example, in 4F2hc or CD98lc gene regulation (and/or as antisense primers in amplification reactions of 4F2hc or CD98lc gene nucleic acid sequences). With respect to 4F2hc and CD98lc gene regulation, such techniques can be used to regulate, for example, hypo- and hyper-thyroidism. Further, such sequences may be used as part of ribozyme and/or triple helix sequences, also useful for 4F2hc and CD98lc gene regulation. Still further, such molecules may be used as components of diagnostic methods whereby, for example, the presence of a particular 4F2hc or CD98lc allele responsible for predisposing to abnormal thyroid hormone transport may be detected.

In addition to the 4F2hc and CD98lc nucleotide sequences described above, full length 4F2hc and CD98lc cDNAs or gene sequences present in the same species or homologues of the 4F2hc and CD98lc genes present in other species can be identified and readily isolated, without undue experimentation, by molecular biological techniques well known in the art. The identification of homologues of 4F2hc and CD98lc in related species can be useful for developing animal model systems more closely related to humans for purposes of drug discovery. For example, expression libraries of cDNAs synthesized from muscle, brain, adipose tissue, placenta, as well as thyroid gland or liver mRNA derived from the organism of interest can be screened using labelled triiodothyronine (T₃), thyroxine (T₄), other iodothyronines, tryptophan, phenylalanine, or subunits that form heterodimers with 4F2hc or CD98lc. Alternatively, such cDNA libraries, or genomic DNA libraries derived from the organism of interest can be screened by hybridization using the nucleotides described herein as hybridization or amplification probes. Furthermore, genes at other genetic loci within the genome that encode proteins which have extensive homology to one or more domains of the 4F2hc or CD98lc gene products can also be identified via similar techniques. In the case of cDNA libraries, such screening techniques can identify clones derived from alternatively spliced transcripts in the same or different species.

Screening can be performed by filter hybridization, using duplicate filters. The labelled probe can contain at least 15-30 base pairs of the 4F2hc or CD98lc nucleotide sequences. The hybridization washing conditions used should be of a lower stringency when the cDNA library is derived from an organism different from the type of organism from which the labelled sequence was derived. For example, hybridization can be performed at 65° C. overnight in Church's buffer (7% SDS, 250 mM NaHPO₄, 2 μM EDTA, 1% BSA). Washes can be done with 2× SSC, 0.1% SDS at 65° C. and then at 0.1×SSC, 0.1% SDS at 65° C.

Low stringency conditions are well known to those of skill in the art, and will vary predictably depending on the specific organisms from which the library and the labelled sequences are derived. For guidance regarding such conditions see, for example, Sambrook et al., 1989, Molecular Cloning, A Laboratory Manual, Cold Springs Harbor Press, N.Y.; and Ausubel et al., 1989, Current Protocols in Molecular Biology, Green Publishing Associates and Wiley Interscience, N.Y.

Alternatively, the labelled 4F2hc or CD98lc nucleotide probe may be used to screen a genomic library derived from the organism of interest, again, using appropriately stringent conditions. The identification and characterization of human genomic clones is helpful for designing diagnostic tests and clinical protocols for treatment of thyroid hormone disorders. For example, sequences derived from regions adjacent to the intron/exon boundaries of the human gene can be used to design primers for use in amplification assays to detect mutations within the exons, introns, splice sites (e.g. splice acceptor and/or donor sites), etc., that can be used in diagnostics.

Further, a 4F2hc or CD98lc gene homologue may be isolated from nucleic acid of the organism of interest by performing PCR using two degenerate oligonucleotide primer pools designed on the basis of amino acid sequences within the 4F2hc and CD98lc gene product disclosed herein. The template for the reaction may be cDNA obtained by reverse transcription of mRNA prepared from, for example, human or non-human cell lines or tissue, such as muscle, brain, adipose tissue, placenta, as well as thyroid gland or liver, known or suspected to express a 4F2hc or CD98lc gene allele.

The PCR product may be subcloned and sequenced to ensure that the amplified sequences represent the sequences of a 4F2hc or CD98lc gene. The PCR fragment may then be used to isolate a full-length cDNA clone by a variety of methods. For example, the amplified fragment may be labelled and used to screen a cDNA library, such as a bacteriophage cDNA library. Alternatively, the labelled fragment may be used to isolate genomic clones via the screening of a genomic library.

PCR technology may also be utilized to isolate full-length cDNA sequences. For example, RNA may be isolated, following standard procedures, from an appropriate cellular or tissue source (i.e., one known, or suspected, to express the 4F2hc or CD98lc gene, such as, for example, muscle, brain, adipose tissue, placenta, as well as thyroid gland or liver). A reverse transcription reaction may be performed on the RNA using an oligonucleotide primer specific for the most 5′ end of the amplified fragment for the priming of first strand synthesis. The resulting RNA/DNA hybrid may then be “tailed” with guanines using a standard terminal transferase reaction, the hybrid may be digested with RNAase H, and second strand synthesis may then be primed with a poly-C primer. Thus, cDNA sequences upstream of the amplified fragment may easily be isolated. For a review of cloning strategies which may be used, see e.g., Sambrook et al., 1989, Molecular Cloning, A Laboratory Manual, Cold Springs Harbor Press, N.Y.; and Ausubel et al., 1989, Current Protocols in Molecular Biology, Green Publishing Associates and Wiley Interscience, N.Y.

The 4F2hc or CD98lc gene sequences may additionally be used to isolate mutant 4F2hc or CD98lc gene alleles. Such mutant alleles may be isolated from individuals either known or proposed to have a genotype that contributes to the symptoms of thyroid hormone disorders. Mutant alleles and mutant allele products may then be utilized in the therapeutic and diagnostic systems described below. Additionally, the 4F2hc or CD98lc gene sequences can be used to detect 4F2hc or CD98lc gene regulatory (e.g., promoter or promoter/enhancer) defects, which can affect thyroid hormone transport.

A cDNA of a mutant 4F2hc or CD98lc gene may be isolated, for example, by using PCR, a technique which is well known to those of skill in the art. In this case, the first cDNA strand may be synthesized by hybridizing an oligo-dT oligonucleotide to mRNA isolated from a tissue known, or suspected, to express a mutant 4F2hc or CD98lc allele in an individual suspected of or known to carry such a mutant allele, and by extending the new strand with reverse transcriptase. The second strand of the cDNA is then synthesized using an oligonucleotide that hybridizes specifically to the 5′ end of the normal gene. Using these two primers, the product is then amplified via PCR, cloned into a suitable vector, and subjected to DNA sequence analysis through methods well known to those of skill in the art. By comparing the DNA sequence of the mutant 4F2hc or CD98lc allele to that of the normal 4F2hc or CD98lc allele, the mutation(s) responsible for the loss or alteration of function of the mutant 4F2hc or CD98lc gene product can be ascertained.

Alternatively, a genomic library can be constructed using DNA obtained from an individual suspected of or known to carry the mutant 4F2hc or CD98lc allele, or a cDNA library can be constructed using RNA from a tissue known, or suspected, to express the mutant 4F2hc or CD98lc allele. The normal 4F2hc or CD98lc gene or any suitable fragment thereof may then be labelled and used as a probe to identify the corresponding mutant 4F2hc or CD98lc allele in such libraries. Clones containing the mutant 4F2hc or CD98lc gene sequences may then be purified and subjected to sequence analysis according to methods well known to those of skill in the art.

Additionally, an expression library can be constructed utilizing cDNA synthesized from, for example, RNA isolated from a tissue known, or suspected, to express a mutant 4F2hc or CD98lc allele in an individual suspected of or known to carry such a mutant allele. In this manner, gene products made by the putatively mutant tissue may be expressed and screened using standard antibody screening techniques in conjunction with antibodies raised against the normal 4F2hc or CD98lc gene product, as described, below, in the sections. (For screening techniques, see, for example, Harlow, E. and Lane, eds., 1988, “Antibodies: A Laboratory Manual”, Cold Spring Harbor Press, Cold Spring Harbor.) Additionally, screening can be accomplished by screening with labelled tri-iodothyronine (T₃), thyroxine (T₄), other iodothyronines, tryptophan, phenylalanine, or subunits that form heterodimers with 4F2hc or CD98lc. In cases where a 4F2hc or CD98lc mutation results in an expressed gene product with altered function (e.g., as a result of a missense or a frameshift mutation), a polyclonal set of antibodies to 4F2hc or CD98lc are likely to cross-react with the mutant 4F2hc or CD98lc gene product. Library clones detected via their reaction with such labelled antibodies can be purified and subjected to sequence analysis according to methods well known to those of skill in the art.

The invention also encompasses nucleotide sequences that encode mutant forms of 4F2hc and CD98lc, peptide fragments of 4F2hc and CD98lc, truncated 4F2hcs and CD98lcs, and 4F2hc and CD98lc fusion proteins. These include, but are not limited to nucleotide sequences encoding mutant 4F2hcs or CD98lcs described in subsequent sections; polypeptides or peptides corresponding to an extracellular domains (ECD), a transmembrane domain (TMD), and/or a cytoplasmic domain (CD) of 4F2hc or CD98lc or portions of these domains; truncated forms of 4F2hc or CD98lc in which one or two of the domains is deleted, e.g., a soluble 4F2hc or CD98lc lacking the TMDs or both the TMDs and CDs, or a truncated, nonfunctional 4F2hc or CD98lc lacking all or a portion of a domain. Nucleotides encoding fusion proteins may include but are not limited to full length 4F2hc or CD98lc, truncated 4F2hc or CD98lc or peptide fragments of 4F2hc or CD98lc fused to an unrelated protein or peptide, such as, for example, a transmembrane sequence which anchors the 4F2hc or CD98lc to the cell membrane; an Ig Fc domain which increases the stability and half life of the resulting fusion protein in the bloodstream; or an enzyme, fluorescent protein, or luminescent protein which can be used as a marker.

The invention also encompasses (a) DNA vectors that contain any of the foregoing 4F2hc or CD98lc coding sequences or their complements (i.e., antisense); (b) DNA expression vectors that contain any of the foregoing 4F2hc or CD98lc coding sequences operatively associated with a regulatory element that directs the expression of the coding sequences; and (c) genetically engineered host cells that contain any of the foregoing 4F2hc or CD98lc coding sequences operatively associated with a regulatory element that directs the expression of the coding sequences in the host cell. As used herein, regulatory elements include but are not limited to inducible and non-inducible promoters, enhancers, operators and other elements known to those skilled in the art that drive and regulate expression. Such regulatory elements include but are not limited to the cytomegalovirus hCMV immediate early gene, the early or late promoters of SV40 adenovirus, the lac system, the trp system, the TAC system, the TRC system, the major operator and promoter regions of phage A, the control regions of fd coat protein, the promoter for 3-phosphoglycerate kinase, the promoters of acid phosphatase, and the promoters of the yeast α-mating factors.

Particular polynucleotides are DNA sequences having any three sequential nucleotides, four sequential nucleotides, five sequential nucleotides, six sequential nucleotides, seven sequential nucleotides, eight sequential nucleotides, nine sequential nucleotides, ten sequential nucleotides, eleven sequential nucleotides, twelve sequential nucleotides, thirteen sequential nucleotides, fourteen sequential nucleotides, fifteen sequential nucleotides, sixteen sequential nucleotides, seventeen sequential nucleotides, eighteen sequential nucleotides, nineteen sequential nucleotides, twenty sequential nucleotides, twenty-one, twenty-two, twenty-three, twenty-four, twenty-five, twenty-six, twenty-seven, twenty-eight, twenty-nine, thirty, thirty-one, thirty-two, thirty-three, thirty-four, thirty-five, thirty-six, thirty-seven, thirty-eight, thirty-nine, forty, forty-one, forty-two, forty-three, forty-four, forty-five, forty-six, forty-seven, forty-eight, forty-nine, fifty, fifty-one, fifty-two, fifty-three, fifty-four, fifty-five, fifty-six, fifty-seven, fifty-eight, fifty-nine, sixty, sixty-one, sixty-two, sixty-three, sixty-four, sixty-five, sixty-six, sixty-seven, sixty-eight, sixty-nine, seventy, seventy-one, seventy-two, seventy-three, seventy-four, seventy-five, seventy-six, seventy-seven, seventy-eight, seventy-nine, eighty, ninety, one-hundred, two hundred, or three hundred or more sequential nucleotides.

The 4F2 Heavy-Chain and CD98 Light-Chain Proteins and Polypeptides

4F2hc and CD98lc proteins, polypeptides and peptide fragments, mutated, truncated or deleted forms of 4F2hc and CD98lc, and 4F2hc and CD98lc fusion proteins can be prepared for a variety of uses, including but not limited to the generation of antibodies, as reagents in diagnostic assays, or the identification of other cellular gene products involved in the regulation of thyroid hormone transport, as reagents in assays for screening for compounds that can be used in the treatment of thyroid hormone disorders, and as pharmaceutical reagents useful in the treatment of thyroid hormone disorders related to the 4F2hc-CD98lc heterodimer.

The 4F2hc amino acid sequences of the invention include the amino acid sequences deduced from the cDNA sequences with Gen Bank Accession numbers: J02939; M17430; M18811; J03569; NM002394. The CD98lc amino acid sequences of the invention include the amino acid sequences deduced from the cDNA sequences with Gen Bank Accession numbers: AF019906; AF077866; AF104032. Further, 4F2hcs and CD98lcs of other species are encompassed by the invention. In fact, any 4F2hc or CD98lc protein encoded by the 4F2hc or CD98lc nucleotide sequences described in the sections above are within the scope of the invention.

The invention also encompasses proteins that are functionally equivalent to 4F2hc or CD98lc encoded by the nucleotide sequences described in the above sections, as judged by any of a number of criteria, including but not limited to the ability to transport tri-iodothyronine (T₃), thyroxine (T₄), other iodothyronines, tryptophan, or phenylalanine, the ability to form heterodimers with 4F2hc or CD98lc, the kinetic properties, the resulting biological effect of T₃ or T₄ transport, e.g., binding to intracellular receptors that activate genes, or change in phenotype when the 4F2hc or CD98lc equivalent is present in an appropriate cell type (such as the amelioration of the hypo- or hyper-thyroid phenotype). Such functionally equivalent 4F2hc and CD98lc proteins include but are not limited to additions or substitutions of amino acid residues within the amino acid sequence encoded by the 4F2hc and CD98lc nucleotide sequences described in the sections above, but which result in a silent change, thus producing a functionally equivalent gene product. Amino acid substitutions may be made on the basis of similarity in polarity, charge, solubility, hydrophobicity, hydrophilicity, and/or the amphipathic nature of the residues involved. For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid. While random mutations can be made to 4F2hc and CD98lc DNA (using random mutagenesis techniques well known to those skilled in the art) and the resulting mutant 4F2hcs and CD98lcs tested for activity, site-directed mutations of the 4F2hc and CD98lc coding sequences can be engineered (using site-directed mutagenesis techniques well known to those skilled in the art) to generate mutant 4F2hcs and CD98lcs with altered function, e.g., different binding affinity for TH, and/or different transporter ability.

For example, identical amino acid residues of a Xenopus laevis form of CD98lc, called IU12, and human forms of CD98lc, called E16 or LAT1, can be aligned so that regions of identity are maintained, whereas the variable residues are altered, e.g., by deletion or insertion of an amino acid residue(s) or by substitution of one or more different amino acid residues. Conservative alterations at the variable positions can be engineered in order to produce a mutant 4F2hc or CD98lc that retains function, e.g., binding affinity for TH or transporter ability, or both. Non-conservative changes can be engineered at these variable positions to alter function, e.g., binding affinity for TH or transporter ability, or both. Alternatively, where alteration of function is desired, deletion or non-conservative alterations of the conserved regions (i.e., identical amino acids) can be engineered. For example, deletion or non-conservative alterations (substitutions or insertions) of a domain within 4F2hc or CD98lc can be engineered to produce a mutant 4F2hc or CD98lc that binds TH, but is transporter-incompetent. The same mutation strategy can also be used to design mutant 4F2hcs and CD98lcs based on the alignment of other non-human homologues of 4F2hc or CD98lc with human homologues of 4F2hc or CD98lc.

Other mutations to the 4F2hc and CD98lc coding sequence can be made to generate 4F2hcs and CD98lcs that are better suited for expression, scale up, etc. in the host cells chosen. For example, cysteine residues can be deleted or substituted with another amino acid in order to eliminate disulfide bridges; N-linked glycosylation sites can be altered or eliminated to achieve, for example, expression of a homogeneous product that is more easily recovered and purified from yeast hosts which are known to hyperglycosylate N-linked sites.

Peptides corresponding to one or more domains of the 4F2hc or CD98lc (e.g., ECD, TMD or CD), truncated or deleted 4F2hcs or CD98lcs (e.g., 4F2hc or CD98lc in which an ECD or a TMD and/or a CD is deleted), as well as fusion proteins in which the full length 4F2hc or CD98lc, a 4F2hc or CD98lc peptide or a truncated 4F2hc or CD98lc is fused to an unrelated protein are also within the scope of the invention and can be designed on the basis of the 4F2hc and CD98lc nucleotide sequences. Such fusion proteins include but are not limited to IgFc fusions which stabilize the 4F2hc or CD98lc protein or peptide and prolong half-life in vivo; or fusions to any amino acid sequence that allows the fusion protein to be anchored to the cell membrane allowing the ECD to be exhibited on the cell surface; or fusions to an enzyme, fluorescent protein, or luminescent protein which provide a marker function.

While the 4F2hc and CD98lc polypeptides and peptides can be chemically synthesized (e.g., see Creighton, 1983, Proteins: Structures and Molecular Principles, W. H. Freeman & Co., N.Y.), large polypeptides derived from the 4F2hc or CD98lc and the full length 4F2hc or CD98lc itself may advantageously be produced by recombinant DNA technology using techniques well known in the art for expressing nucleic acid containing 4F2hc or CD98lc gene sequences and/or coding sequences. Such methods can be used to construct expression vectors containing the 4F2hc or CD98lc nucleotide sequences and appropriate transcriptional and translational control signals. These methods include, for example, in vitro recombinant DNA techniques, synthetic techniques, and in vivo genetic recombination. See, for example, the techniques described in Sambrook et al., 1989, Molecular Cloning, A Laboratory Manual, Cold Springs Harbor Press, N.Y.; and Ausubel et al., 1989, Current Protocols in Molecular Biology, Green Publishing Associates and Wiley Interscience, N.Y. Alternatively, RNA capable of encoding 4F2hc or CD98lc nucleotide sequences may be chemically synthesized using, for example, synthesizers. See, for example, the techniques described in “Oligonucleotide Synthesis”, 1984, Gait, M. J. ed., In Press, Oxford.

A variety of host-expression vector systems may be utilized to express the 4F2hc or CD98lc nucleotide sequences of the invention. Where the 4F2hc or CD98lc peptide or polypeptide is soluble (e.g., 4F2hc or CD98lc peptides corresponding to an ECD; truncated or deleted 4F2hc or CD98lc in which a TMD and/or a CD are deleted), the peptide or polypeptide can be recovered from the culture, i.e., from the host cell in cases where the 4F2hc or CD98lc peptide or polypeptide is not secreted, and from the culture media in cases where the 4F2hc or CD98lc peptide or polypeptide is secreted by the cells. However, the expression systems also encompass engineered host cells that express 4F2hc or CD98lc or functional equivalents in situ, i.e., anchored in the cell membrane. Purification or enrichment of 4F2hc or CD98lc from such expression systems can be accomplished using appropriate detergents and lipid micelles and methods well known to those skilled in the art. However, such engineered host cells themselves may be used in situations where it is important not only to retain the structural and functional characteristics of 4F2hc or CD98lc, but to assess biological activity, e.g., in drug screening assays.

The expression systems that may be used for purposes of the invention include but are not limited to microorganisms such as bacteria (e.g., E. coli, B. subtilis) transformed with recombinant bacteriophage DNA, plasmid DNA or cosmid DNA expression vectors containing 4F2hc or CD98lc nucleotide sequences; yeast (e.g., Saccharomyces, Pichia) transformed with recombinant yeast expression vectors containing the 4F2hc or CD98lc nucleotide sequences; insect cell systems (e.g., SF9) infected with recombinant virus expression vectors (e.g., baculovirus) containing the 4F2hc or CD98lc sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing 4F2hc or CD98lc nucleotide sequences; or mammalian cell systems (e.g., COS, CHO, BHK, 293, 3T3) harboring recombinant expression constructs containing promoters derived from the genome of mammalian cells (e.g., metallothionein promoter) or from mammalian viruses (e.g., the adenovirus late promoter; the vaccinia virus 7.5K promoter).

In bacterial systems, a number of expression vectors may be advantageously selected depending upon the use intended for the 4F2hc or CD98lc gene product being expressed. For example, when a large quantity of such a protein is to be produced, for the generation of pharmaceutical compositions of 4F2hc or CD98lc protein, or for raising antibodies to either the 4F2hc or CD98lc protein, for example, vectors which direct the expression of high levels of fusion protein products that are readily purified may be desirable. Such vectors include, but are not limited, to the E. coli expression vector pUR278 (Ruther et al., 1983, EMBO J. 2:1791), in which the 4F2hc or CD98lc coding sequence may be ligated individually into the vector in frame with the lacZ coding region so that a fusion protein is produced; pIN vectors (Inouye & Inouye, 1985, Nucleic Acids Res 13:3101-3109; Van Heeke & Schuster, 1989, J Biol Chem 264:5503-5509); and the like. pGEX vectors may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhidrosis virus (AcNPV) is used as a vector to express foreign genes. The virus grows in Spodoptera frugiperda cells. The 4F2hc or CD98lc gene coding sequence may be cloned individually into nonessential regions (for example the polyhedrin gene) of the virus and placed under control of an AcNPV promoter (for example the polyhedrin promoter). Successful insertion of 4F2hc or CD98lc gene coding sequence will result in inactivation of the polyhedrin gene and production of non-occluded recombinant virus, (i.e., virus lacking the proteinaceous coat coded for by the polyhedrin gene). These recombinant viruses are then used to infect Spodoptera frugiperda cells in which the inserted gene is expressed. (E.g., see Smith et al., 1983, J Virol 46: 584; Smith, U.S. Pat. No. 4,215,051).

In mammalian host cells, a number of viral-based expression systems may be utilized. In cases where an adenovirus is used as an expression vector, the 4F2hc or CD98lc nucleotide sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the 4F2hc or CD98lc gene product in infected hosts. (E.g., See Logan & Shenk, 1984, Proc Natl Acad Sci USA 81:3655-3659). Specific initiation signals may also be required for efficient translation of inserted 4F2hc or CD98lc nucleotide sequences. These signals include the ATG initiation codon and adjacent sequences. In cases where an entire 4F2hc or CD98lc gene or cDNA, including its own initiation codon and adjacent sequences, is inserted into the appropriate expression vector, no additional translational control signals may be needed. However, in cases where only a portion of the 4F2hc or CD98lc coding sequence is inserted, exogenous translational control signals, including, perhaps, the ATG initiation codon, must be provided. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (See Bittner et al., 1987, Methods in Enzymol 153:516-544).

In addition, a host cell strain may be chosen, which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells, which possess the cellular machinery for proper processing of the primary transcript, glycosylation, and phosphorylation of the gene product, may be used. Such mammalian host cells include but are not limited to CHO, VERO, BHK, HeLa, COS, MDCK, 293, 3T3, WI38, in addition to muscle, brain, adipose tissue, placenta, as well as thyroid gland or liver cell lines.

For long-term, high-yield production of recombinant proteins, stable expression is preferred. For example, cell lines, which stably express the 4F2hc or CD98lc sequences described above, may be engineered. Rather than using expression vectors which contain viral origins of replication, host cells can be transformed with DNA controlled by appropriate expression control elements (e.g., promoter, enhancer sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines, which express the 4F2hc and CD98lc gene product. Such engineered cell lines may be particularly useful in screening and evaluation of compounds that affect the endogenous activity of the 4F2hc and CD98lc gene product.

A number of selection systems may be used, including but not limited to the herpes simplex virus thymidine kinase (Wigler, et al., 1977, Cell 11:223), hypoxanthine-guanine phosphoribosyltransferase (Szybalska & Szybalski, 1962, Proc Natl Acad Sci USA 48:2026), and adenine phosphoribosyltransferase (Lowy, et al., 1980, Cell 22:817) genes can be employed in tk-, hgprt- or aprt-cells, respectively. Also, antimetabolite resistance can be used as the basis of selection for the following genes: dhfr, which confers resistance to methotrexate (Wigler, et al., 1980, Proc Natl Acad Sci USA 77:3567; O'Hare, et al., 1981, Proc Natl Acad Sci USA 78:1527); gpt, which confers resistance to mycophenolic acid (Mulligan & Berg, 1981, Proc Natl Acad Sci USA 78:2072); neo, which confers resistance to the aminoglycoside G-418 (Colberre-Garapin, et al., 1981, J Mol Biol 150:1); and hygro, which confers resistance to hygromycin (Santerre, et al., 1984, Gene 30:147).

Alternatively, any fusion protein may be readily purified by utilizing an antibody specific for the fusion protein being expressed. For example, a system described by Janknecht et al. allows for the ready purification of non-denatured fusion proteins expressed in human cell lines (Janknecht, et al., 1991, Proc Natl Acad Sci USA 88: 8972-8976). In this system, the gene of interest is subcloned into a vaccinia recombination plasmid such that the gene's open reading frame is translationally fused to an amino-terminal tag consisting of six histidine residues. Extracts from cells infected with recombinant vaccinia virus are loaded onto Ni²⁺ nitriloacetic acid-agarose columns and histidine-tagged proteins are selectively eluted with imidazole-containing buffers.

The 4F2hc and CD98lc gene products can also be expressed/coexpressed in transgenic animals. Animals of any species, including, but not limited to, mice, rats, rabbits, guinea pigs, pigs, micro-pigs, goats, and non-human primates, e.g., baboons, monkeys, and chimpanzees may be used to generate 4F2hc and CD98lc transgenic animals.

Particular polypeptides are amino acid sequences having any three sequential residues, four sequential residues, five sequential residues, six sequential residues, seven sequential residues, eight sequential residues, nine sequential residues, ten sequential residues, eleven sequential residues, twelve sequential residues, thirteen sequential residues, fourteen sequential residues, fifteen sequential residues, sixteen sequential residues, seventeen sequential residues, eighteen sequential residues, nineteen sequential residues, twenty sequential residues, twenty-one, twenty-two, twenty-three, twenty-four, twenty-five, twenty-six, twenty-seven, twenty-eight, twenty-nine, thirty, forty, fifty, sixty, seventy, eighty, ninety, or more sequential residues.

Screening Assays for Compounds that Modulate 4F2hc or CD98lc Expression or Activity

The following assays are designed to identify compounds that interact with 4F2hc or CD98lc (including but not limited to an ECD or a CD and/or a TMD of 4F2hc or CD98lc), compounds that interact with transmembrane or intracellular proteins that interact with 4F2hc or CD98lc (including but not limited to a TMD or a CD of 4F2hc or CD98lc), and compounds that modulate the activity of the 4F2hc or CD98lc gene (i.e., modulate the level of 4F2hc or CD98lc gene expression) or modulate the level of 4F2hc or CD98lc. Assays may additionally be utilized that identify compounds that bind to 4F2hc or CD98lc gene regulatory sequences (e.g., promoter sequences) and that may modulate 4F2hc or CD98lc gene expression.

The compounds which may be screened in accordance with the invention include, but are not limited to peptides, antibodies and fragments thereof, and other organic compounds (e.g., peptidomimetics) that bind to an ECD of 4F2hc or CD98lc and either mimic the activity triggered by the natural thyroid hormone or inhibit the activity triggered by the natural thyroid hormone; as well as peptides, antibodies or fragments thereof, and other organic compounds that mimic an ECD of 4F2hc or CD98lc (or a portion thereof) and bind to and “neutralize” the natural thyroid hormone.

Such compounds may include, but are not limited to, peptides such as, for example, soluble peptides, including but not limited to members of random peptide libraries; (see, e.g., Lam, K. S. et al., 1991, Nature 354:82-84; Houghten, R. et al., 1991, Nature 354:84-86), and combinatorial chemistry-derived molecular libraries made of D- and/or L-configuration amino acids, phosphopeptides (including, but not limited to, members of random or partially degenerate, directed phosphopeptide libraries; see, e.g., Songyang, Z. et al., 1993, Cell 72:767-778), antibodies (including, but not limited to, polyclonal, monoclonal, humanized, anti-idiotypic, chimeric or single chain antibodies, and FAb, F(ab′)₂ and FAb expression library fragments, and epitope-binding fragments thereof), and small organic or inorganic molecules.

Other compounds which can be screened in accordance with the invention include but are not limited to small organic molecules that are able to gain entry into the appropriate cell (e.g., in muscle, brain, adipose tissue, placenta, as well as thyroid gland or liver) and affect the expression of the 4F2hc or CD98lc gene or some other gene involved in the 4F2hc or CD98lc signal transduction pathway (e.g., by interacting with the regulatory region or transcription factors involved in gene expression); or such compounds that affect the activity of 4F2hc or CD98lc (e.g., by inhibiting or enhancing the transport activity of a CD), or the activity of some other transmembrane or intracellular factor involved in the 4F2hc or CD98lc signal transduction pathway.

Computer modeling and searching technologies permit identification of compounds, or the improvement of already identified compounds, that can modulate 4F2hc or CD98lc expression or activity. Having identified such a compound or composition, the active sites or regions are identified. Such active sites might typically be ligand binding sites, such as the interaction domains of TH with 4F2hc or CD98lc itself. The active site can be identified using methods known in the art including, for example, from the amino acid sequences of peptides, from the nucleotide sequences of nucleic acids, or from study of complexes of the relevant compound or composition with its ligand. In the latter case, chemical or X-ray crystallographic methods can be used to find the active site by finding where on the factor the complexed ligand is found. Next, the three dimensional geometric structure of the active site is determined. This can be done by known methods, including X-ray crystallography, which can determine a complete molecular structure. On the other hand, solid or liquid phase NMR can be used to determine certain intra-molecular distances. Any other experimental method of structure determination can be used to obtain partial or complete geometric structures. The geometric structures may be measured with a complexed ligand, natural or artificial, which may increase the accuracy of the active site structure determined.

If an incomplete or insufficiently accurate structure is determined, the methods of computer based numerical modeling can be used to complete the structure or improve its accuracy. Any recognized modeling method may be used, including parameterized models specific to particular biopolymers such as proteins or nucleic acids, molecular dynamics models based on computing molecular motions, statistical mechanics models based on thermal ensembles, or combined models. For most types of models, standard molecular force fields, representing the forces between constituent atoms and groups, are necessary, and can be selected from force fields known in physical chemistry. The incomplete or less accurate experimental structures can serve as constraints on the complete and more accurate structures computed by these modeling methods.

Finally, having determined the structure of the active site, either experimentally, by modeling, or by a combination, candidate modulating compounds can be identified by searching databases containing compounds along with information on their molecular structure. Such a search seeks compounds having structures that match the determined active site structure and that interact with the groups defining the active site. Such a search can be manual, but is preferably computer assisted. These compounds found from this search are potential 4F2hc or CD98lc modulating compounds.

Alternatively, these methods can be used to identify improved modulating compounds from an already known modulating compound or ligand. The composition of the known compound can be modified and the structural effects of modification can be determined using the experimental and computer modeling methods described above applied to the new composition. The altered structure is then compared to the active site structure of the compound to determine if an improved fit or interaction results. In this manner systematic variations in composition, such as by varying side groups, can be quickly evaluated to obtain modified modulating compounds or ligands of improved specificity or activity.

Further experimental and computer modeling methods useful to identify modulating compounds based upon identification of the active sites of TH, 4F2hc, CD98lc, and related transduction and transcription factors will be apparent to those of skill in the art.

Examples of molecular modeling systems are the CHARMM and QUANTA programs (Polygen Corporation, Waltham, Mass.). CHARMm performs the energy minimization and molecular dynamics functions. QUANTA performs the construction, graphic modeling and analysis of molecular structure. QUANTA allows interactive construction, modification, visualization, and analysis of the behavior of molecules with each other.

A number of articles review computer modeling of drugs interactive with specific-proteins, such as Rotivinen, et al., 1988, Acta Pharmaceutical Fennica 97:159-166; Ripka, 1988, New Scientist 54-57; McKinaly and Rossmann, 1989, Annu Rev Pharmacol Toxicol 29:111-122; Perry and Davies, OSAR: Quantitative Structure-Activity Relationships in Drug Design pp. 189-193 (Alan R. Liss, Inc. 1989); Lewis and Dean, 1989 Proc R Soc Lond 236:125-140 and 141-162; and, with respect to a model receptor for nucleic acid components, Askew, et al., 1989, J Am Chem Soc 111:1082-1090. Other computer programs that screen and graphically depict chemicals are available from companies such as BioDesign, Inc. (Pasadena, Calif.), Allelix, Inc. (Mississauga, Ontario, Canada), and Hypercube, Inc. (Cambridge, Ontario). Although these are primarily designed for application to drugs specific to particular proteins, they can be adapted to design of drugs specific to regions of DNA or RNA, once that region is identified.

Although described above with reference to design and generation of compounds which could alter binding, one could also screen libraries of known compounds, including natural products or synthetic chemicals, and biologically active materials, including proteins, for compounds which are inhibitors or activators.

Compounds identified via assays such as those described herein may be useful, for example, in elaborating the biological function of the 4F2hc and CD98lc gene product, and for ameliorating thyroid hormone disorders.

In Vitro Screening Assays for Compounds that Bind to 4F2hc or CD98lc

In vitro systems may be designed to identify compounds capable of interacting with (e.g., binding to) 4F2hc or CD98lc (including but not limited to an ECD or a CD or a TMD of 4F2hc or CD98lc). Compounds identified may be useful, for example, in modulating the activity of wild type and/or mutant 4F2hc or CD98lc gene products; may be useful in elaborating the biological function of 4F2hc or CD98lc; may be utilized in screens for identifying compounds that disrupt normal 4F2hc or CD98lc interactions; or may in themselves disrupt such interactions.

The principle of the assays used to identify compounds that bind to 4F2hc or CD98lc involves preparing a reaction mixture of 4F2hc or CD98lc and the test compound under conditions and for a time sufficient to allow the components to interact and bind, thus forming a complex which can be removed and/or detected in the reaction mixture. The 4F2hc or CD98lc species used can vary depending upon the goal of the screening assay. For example, where compounds that mimic the natural hormone are sought, the full length 4F2hc or CD98lc, or a soluble truncated 4F2hc or CD98lc, e.g., in which a TMD and/or a CD is deleted from the molecule, a peptide corresponding to an ECD or a fusion protein containing the 4F2hc or CD98lc ECD fused to a protein or polypeptide that affords advantages in the assay system (e.g., labeling, isolation of the resulting complex, etc.) can be utilized. Where compounds that interact with a CD are sought to be identified, peptides corresponding to the 4F2hc or CD98lc CD and fusion proteins containing the 4F2hc or CD98lc CD can be used.

The screening assays can be conducted in a variety of ways. For example, one method to conduct such an assay would involve anchoring the 4F2hc or CD98lc protein, polypeptide, peptide or fusion protein or the test substance onto a solid phase and detecting 4F2hc or CD98lc/test compound complexes anchored on the solid phase at the end of the reaction. In one embodiment of such a method, the 4F2hc or CD98lc reactant may be anchored onto a solid surface, and the test compound, which is not anchored, may be labelled, either directly or indirectly.

In practice, microtiter plates may conveniently be utilized as the solid phase. The anchored component may be immobilized by non-covalent or covalent attachments. Non-covalent attachment may be accomplished by simply coating the solid surface with a solution of the protein and drying. Alternatively, an immobilized antibody, preferably a monoclonal antibody, specific for the protein to be immobilized may be used to anchor the protein to the solid surface. The surfaces may be prepared in advance and stored.

In order to conduct the assay, the nonimmobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously nonimmobilized component is pre-labelled, the detection of label immobilized on the surface indicates that complexes were formed. Where the previously nonimmobilized component is not pre-labelled, an indirect label can be used to detect complexes anchored on the surface; e.g., using a labelled antibody specific for the previously nonimmobilized component (the antibody, in turn, may be directly labelled or indirectly labelled with a labelled anti-Ig antibody).

Alternatively, a reaction can be conducted in a liquid phase, the reaction products separated from unreacted components, and complexes detected; e.g., using an immobilized antibody specific for 4F2hc or CD98lc protein, polypeptide, peptide or fusion protein or the test compound to anchor any complexes formed in solution, and a labelled antibody specific for the other component of the possible complex to detect anchored complexes.

Alternatively, cell-based assays, membrane vesicle-based assays, and membrane fraction-based assays can be used to identify compounds that interact with 4F2hc or CD98lc. To this end, cell lines that express 4F2hc or CD98lc, or cell lines (e.g., COS cells, CHO cells, fibroblasts, etc.) that have been genetically engineered to express 4F2hc or CD98lc (e.g., by transfection or transduction of 4F2hc or CD98lc DNA) can be used. Interaction of the test compound with, for example, an ECD of 4F2hc or CD98lc expressed by the host cell can be determined by comparison or competition with the natural hormone.

Assays for Transmembrane or Intracellular Proteins that Interact with 4F2hc or CD98lc

Any method suitable for detecting protein-protein interactions may be employed for identifying transmembrane or intracellular proteins that interact with 4F2hc or CD98lc. Among the traditional methods which may be employed are co-immunoprecipitation, crosslinking and co-purification through gradients or chromatographic columns of cell lysates or proteins obtained from cell lysates and 4F2hc or CD98lc or to identify proteins in the lysate that interact with 4F2hc or CD98lc. For these assays, the 4F2hc or CD98lc component used can be a full length 4F2hc or CD98lc, a soluble derivative lacking the membrane-anchoring region (e.g., a truncated 4F2hc or CD98lc in which the TMD is deleted resulting in a truncated molecule containing the ECD fused to the CD), a peptide corresponding to the CD or a fusion protein containing the CD of 4F2hc or CD98lc. Once isolated, such a transmembrane or intracellular protein can be identified and can, in turn, be used, in conjunction with standard techniques, to identify proteins with which it interacts. For example, at least a portion of the amino acid sequence of an intracellular protein which interacts with 4F2hc or CD98lc can be ascertained using techniques well known to those of skill in the art, such as via the Edman degradation technique. (See, e.g., Creighton, 1983, “Proteins: Structures and Molecular Principles”, W.H. Freeman & Co., N.Y., pp. 34-49). The amino acid sequence obtained may be used as a guide for the generation of oligonucleotide mixtures that can be used to screen for gene sequences encoding such intracellular proteins. Screening may be accomplished, for example, by standard hybridization or PCR techniques. Techniques for the generation of oligonucleotide mixtures and the screening are well known. (See, e.g., Ausubel et al., 1989, Current Protocols in Molecular Biology, Green Publishing Associates and Wiley Interscience, N.Y., and Innis, M. et al., PCR Protocols: A Guide to Methods and Applications, 1990, eds. Academic Press, Inc., New York).

Additionally, methods may be employed which result in the simultaneous identification of genes, which encode the transmembrane or intracellular proteins interacting with 4F2hc or CD98lc. These methods include, for example, probing expression libraries, in a manner similar to the well known technique of antibody probing of λgt11 libraries, using labelled 4F2hc or CD98lc protein, or a 4F2hc or CD98lc polypeptide, peptide or fusion protein, e.g., a 4F2hc or CD98lc polypeptide or domain fused to a marker (e.g., an enzyme, fluor, luminescent protein, or dye), or an IgFc domain.

One method, which detects protein interactions in vivo, the two-hybrid system, is described in detail for illustration only and not by way of limitation. One version of this system has been described (Chien et al., 1991, Proc Natl Acad Sci USA, 88:9578-9582) and is commercially available from Clontech (Palo Alto, Calif.).

Briefly, utilizing such a system, plasmids are constructed that encode two hybrid proteins: one plasmid consists of nucleotides encoding the DNA-binding domain of a transcription activator protein fused to a 4F2hc or CD98lc nucleotide sequence encoding 4F2hc or CD98lc, a 4F2hc or CD98lc polypeptide, peptide or fusion protein, and the other plasmid consists of nucleotides encoding the transcription activator protein's activation domain fused to a cDNA encoding an unknown protein which has been recombined into this plasmid as part of a cDNA library. The DNA-binding domain fusion plasmid and the cDNA library are transformed into a strain of the yeast Saccharomyces cerevisiae that contains a reporter gene (e.g., HBS or lacZ) whose regulatory region contains the transcription activator's binding site. Either hybrid protein alone cannot activate transcription of the reporter gene: the DNA-binding domain hybrid cannot because it does not provide activation function and the activation domain hybrid cannot because it cannot localize to the activator's binding sites. Interaction of the two hybrid proteins reconstitutes the functional activator protein and results in expression of the reporter gene, which is detected by an assay for the reporter gene product.

The two-hybrid system or related methodology may be used to screen activation domain libraries for proteins that interact with the “bait” gene product. By way of example, and not by way of limitation, 4F2hc or CD98lc may be used as the bait gene product. Total genomic or cDNA sequences are fused to the DNA encoding an activation domain. This library and a plasmid encoding a hybrid of a bait 4F2hc or CD98lc gene product fused to the DNA-binding domain are cotransformed into a yeast reporter strain, and the resulting transformants are screened for those that express the reporter gene. For example, and not by way of limitation, a bait 4F2hc or a CD98lc gene sequence, such as the open reading frame of 4F2hc or CD98lc (or a domain of 4F2hc or CD98lc), can be cloned into a vector such that it is translationally fused to the DNA encoding the DNA-binding domain of the GAL4 protein. These colonies are purified and the library plasmids responsible for reporter gene expression are isolated. DNA sequencing is then used to identify the proteins encoded by the library plasmids.

A cDNA library of the cell line from which proteins that interact with bait 4F2hc or CD98lc gene product are to be detected can be made using methods routinely practiced in the art. According to the particular system described herein, for example, the cDNA fragments can be inserted into a vector such that they are translationally fused to the transcriptional activation domain of GAL4. This library can be co-transformed along with the bait 4F2hc or CD98lc gene-GAL4 fusion plasmid into a yeast strain, which contains a lacZ gene driven by a promoter, which contains GAL4 activation sequence. A cDNA encoded protein, fused to GAL4 transcriptional activation domain that interacts with bait 4F2hc or CD98lc gene product will reconstitute an active GAL4 protein and thereby drive expression of the HIS3 gene. Colonies that express HIS3 can be detected by their growth on Petri dishes containing semi-solid agar based media lacking histidine. The cDNA can then be purified from these strains, and used to produce and isolate the bait 4F2hc or CD98lc gene-interacting protein using techniques routinely practiced in the art.

Assays for Compounds that Interfere with 4F2hc or CD98lc/Macromolecule Interaction

The macromolecules that interact with 4F2hc or CD98lc are referred to, for purposes of this discussion, as “ligands”. These ligands are likely to be involved in the 4F2hc and CD98lc signal transduction pathway, and therefore, in the role of 4F2hc and CD98lc in thyroid hormone transport. Therefore, it is desirable to identify compounds that interfere with or disrupt the interaction of such ligands with 4F2hc or CD98lc which may be useful in regulating the activity of 4F2hc or CD98lc and control thyroid hormone disorders associated with 4F2hc or CD98lc activity.

The basic principle of the assay systems used to identify compounds that interfere with the interaction between 4F2hc or CD98lc and its ligand or ligands involves preparing a reaction mixture containing 4F2hc or CD98lc protein, polypeptide, peptide or fusion protein, as described in the sections above, and the ligand under conditions and for a time sufficient to allow the two to interact and bind, thus forming a complex. In order to test a compound for inhibitory activity, the reaction mixture is prepared in the presence and absence of the test compound. The test compound may be initially included in the reaction mixture, or may be added at a time subsequent to the addition of the 4F2hc or CD98lc moiety and its ligand. Control reaction mixtures are incubated without the test compound or with a placebo. The formation of any complexes between the 4F2hc or CD98lc moiety and the ligand is then detected. The formation of a complex in the control reaction, but not in the reaction mixture containing the test compound, indicates that the compound interferes with the interaction of the 4F2hc or CD98lc and the interactive ligand. Additionally, complex formation within reaction mixtures containing the test compound and normal 4F2hc or CD98lc protein may also be compared to complex formation within reaction mixtures containing the test compound and a mutant 4F2hc or CD98lc. This comparison may be important in those cases wherein it is desirable to identify compounds that disrupt interactions of mutant but not normal 4F2hcs or CD98lcs.

The assay for compounds that interfere with the interaction of the 4F2hc or CD98lc and ligands can be conducted in a heterogeneous or homogeneous format. Heterogeneous assays involve anchoring either the 4F2hc or CD98lc moiety product or the ligand onto a solid phase and detecting complexes anchored on the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction by competition can be identified by conducting the reaction in the presence of the test substance; i.e., by adding the test substance to the reaction mixture prior to or simultaneously with the 4F2hc or CD98lc moiety and interactive ligand. Alternatively, test compounds that disrupt preformed complexes, e.g. compounds with higher binding constants that displace one of the components from the complex, can be tested by adding the test compound to the reaction mixture after complexes have been formed. The various formats are described briefly below.

In a heterogeneous assay system, either the 4F2hc or CD98lc moiety, or the interactive binding partner, is anchored onto a solid surface, while the non-anchored species is labelled, either directly or indirectly. In practice, microtiter plates are conveniently utilized. The anchored species may be immobilized by non-covalent or covalent attachments. Non-covalent attachment may be accomplished simply by coating the solid surface with a solution of the 4F2hc or CD98lc gene product or ligand and drying. Alternatively, an immobilized antibody specific for the species to be anchored may be used to anchor the species to the solid surface. The surfaces may be prepared in advance and stored.

In order to conduct the assay, the partner of the immobilized species is exposed to the coated surface with or without the test compound. After the reaction is complete, unreacted components are removed (e.g., by washing) and any complexes formed will remain immobilized on the solid surface. The detection of complexes anchored on the solid surface can be accomplished in a number of ways. Where the non-immobilized species is pre-labelled, the detection of label immobilized on the surface indicates that complexes were formed. Where the non-immobilized species is not pre-labelled, an indirect label can be used to detect complexes anchored on the surface; e.g., using a labelled antibody specific for the initially non-immobilized species (the antibody, in turn, may be directly labelled or indirectly labelled with a labelled anti-Ig antibody). Depending upon the order of addition of reaction components, test compounds which inhibit complex formation or which disrupt preformed complexes can be detected.

Alternatively, the reaction can be conducted in a liquid phase in the presence or absence of the test compound, the reaction products separated from unreacted components, and complexes detected; e.g., using an immobilized antibody specific for one of the binding components to anchor any complexes formed in solution, and a labelled antibody specific for the other partner to detect anchored complexes. Again, depending upon the order of addition of reactants to the liquid phase, test compounds which inhibit complex or which disrupt preformed complexes can be identified.

In an alternate embodiment of the invention, a homogeneous assay can be used. In this approach, a preformed complex of the 4F2hc or CD98lc moiety and the interactive ligand is prepared in which either the 4F2hc or CD98lc moiety or its ligand is labelled, but the signal generated by the label is quenched due to formation of the complex (see, e.g., U.S. Pat. No. 4,109,496 by Rubenstein which utilizes this approach for immunoassays). The addition of a test substance that competes with and displaces one of the species from the preformed complex will result in the generation of a signal above background. In this way, test substances, which disrupt the 4F2hc or CD98lc/ligand interaction, can be identified.

In a particular embodiment, a 4F2hc or CD98lc fusion can be prepared for immobilization. For example, the 4F2hc or CD98lc or a peptide fragment, e.g., corresponding to the ECD or CD, can be fused to a glutathione-S-transferase (GST) gene using a fusion vector, such as pGEX-5X-1, in such a manner that its binding activity is maintained in the resulting fusion protein. The interactive ligand can be purified and used to raise a monoclonal antibody, using methods routinely practiced in the art. This antibody can be labelled with the radioactive isotope ¹²⁵I, for example, by methods routinely practiced in the art. In a heterogeneous assay, e.g., the GST-4F2hc or -CD98lc fusion protein can be anchored to glutathione-agarose beads. The interactive ligand can then be added in the presence or absence of the test compound in a manner that allows interaction and binding to occur. At the end of the reaction period, unbound material can be washed away, and the labelled monoclonal antibody can be added to the system and allowed to bind to the complexed components. The interaction between the 4F2hc or CD98lc and the interactive ligand can be detected by measuring the amount of radioactivity that remains associated with the glutathione-agarose beads. A successful inhibition of the interaction by the test compound will result in a decrease in measured radioactivity.

Alternatively, the GST-4F2hc or -CD98lc fusion protein and the interactive ligand can be mixed together in liquid in the absence of the solid glutathione-agarose beads. The test compound can be added either during or after the species are allowed to interact. This mixture can then be added to the glutathione-agarose beads and unbound material is washed away. Again, the extent of inhibition of the 4F2hc or CD98lc/ligand interaction can be detected by adding the labelled antibody and measuring the radioactivity associated with the beads.

In another embodiment of the invention, these same techniques can be employed using peptide fragments that correspond to the binding domains of 4F2hc or CD98lc and/or the interactive ligand (in cases where the ligand is a protein), in place of one or both of the full length proteins. Any number of methods routinely practiced in the art can be used to identify and isolate the binding sites. These methods include, but are not limited to, mutagenesis of the gene encoding one of the proteins and screening for disruption of binding in a co-immunoprecipitation assay. Compensating mutations in the gene encoding the second species in the complex can then be selected. Sequence analysis of the genes encoding the respective proteins will reveal the mutations that correspond to the region of the protein involved in interactive binding. Alternatively, one protein can be anchored to a solid surface using methods described above, and allowed to interact with and bind to its labelled ligand, which has been treated with a proteolytic enzyme, such as trypsin. After washing, a short, labelled peptide comprising the binding domain may remain associated with the solid material, which can be isolated and identified by amino acid sequencing. Also, once the gene coding for the interactive ligand is obtained, short gene segments can be engineered to express peptide fragments of the protein, which can then be tested for binding activity and purified or synthesized.

For example, and not by way of limitation, a 4F2hc or CD98lc gene product can be anchored to a solid material as described, above, by making a GST-4F2hc or CD98lc fusion protein and allowing it to bind to glutathione agarose beads. The interactive ligand can be labelled with a radioactive isotope, such as ³⁵S, and cleaved with a proteolytic enzyme such as trypsin. Cleavage products can then be added to the anchored GST-4F2hc or -CD98lc fusion protein and allowed to bind. After washing away unbound peptides, labelled bound material, representing the interactive ligand binding domain, can be eluted, purified, and analyzed for amino acid sequence by well-known methods. Peptides so identified can be produced synthetically or fused to appropriate facilitative proteins using recombinant DNA technology.

Assays for Identification of Compounds that Ameliorate Thyroid Hormone Disorders

Compounds, including but not limited to binding compounds identified via assay techniques such as those described in the preceding sections above can be tested for the ability to ameliorate thyroid hormone disorders. The assays described above can identify compounds which affect 4F2hc or CD98lc activity (e.g., compounds that bind to 4F2hc or CD98lc, inhibit binding of the natural hormone, and either activate signal transduction (e.g., increase transport) or block activation (e.g., decrease transport), and compounds that bind to a natural ligand of 4F2hc or CD98lc and neutralize ligand activity; or compounds that affect 4F2hc or CD98lc gene activity (by affecting 4F2hc or CD98lc gene expression, including molecules, e.g., proteins or small organic molecules, that affect or interfere with splicing events so that expression of the full length 4F2hc or CD98lc can be modulated). However, it should be noted that the assays described can also identify compounds that modulate 4F2hc or CD98lc signal transduction (e.g., compounds that affect upstream or downstream signaling events). The identification and use of such compounds which affect another step in the 4F2hc or CD98lc signal transduction pathway in which the 4F2hc-CD98lc gene product is involved and, by affecting this same pathway may modulate the effect of 4F2hc or CD98lc on the development of thyroid hormone disorders are within the scope of the invention. Such compounds can be used as part of a therapeutic method for the amelioration of thyroid hormone disorders.

The invention encompasses cell-based and animal model-based assays for the identification of compounds exhibiting such an ability to ameliorate thyroid hormone disorders. Cell-based assays, membrane vesicle-based assays, and membrane fraction-based assays are envisioned. Such cell-based assay systems can be used as the “gold standard” to assay for purity and potency of the candidate compound.

Cell-based systems can be used to identify compounds that may act to ameliorate thyroid hormone disorders. Such cell systems can include, for example, recombinant or non-recombinant cells, such as cell lines, which express the 4F2hc or CD98lc genes. For example, muscle, brain, adipose tissue, placenta, as well as thyroid gland or liver cells, or cell lines derived from these tissues can be used. Additionally, frog oocytes in which cDNAs are functionally expressed can be tested for transmembrane hormone transport. Furthermore, expression host cells (e.g., COS cells, CHO cells, fibroblasts) genetically engineered to express a functional 4F2hc or CD98lc and to respond to activation by the natural hormone, e.g., as measured by a chemical or phenotypic change, induction of another host cell gene, or change in transport activity, etc., can be used as an end point in the assay.

In utilizing such cell systems, cells may be exposed to a compound suspected of exhibiting an ability to ameliorate thyroid hormone disorders, at a sufficient concentration and for a time sufficient to elicit a cellular phenotype associated with an amelioration of thyroid hormone disorders in the exposed cells. After exposure, the cells can be assayed to measure alterations in the expression of the 4F2hc or CD98lc gene, e.g., by assaying cell lysates for 4F2hc or CD98lc mRNA transcripts (e.g., by Northern analysis) or for 4F2hc or CD98lc protein expressed in the cell; compounds which regulate or modulate expression of the 4F2hc or CD98lc gene are good candidates as therapeutics. Alternatively, the cells are examined to determine whether one or more cellular phenotypes associated with thyroid hormone disorders has been altered to resemble cellular phenotype associated with normal thyroid hormone transport. Still further, the expression or activity of components of the signal transduction pathway of which 4F2hc or CD98lc is a part, or the activity of the 4F2hc or CD98lc signal transduction pathway itself can be assayed, e.g., by measuring transport activity.

For example, after exposure, the cell lysates can be assayed for the presence of host cell proteins, as compared to lysates derived from unexposed control cells. The ability of a test compound to inhibit recruitment of host cell proteins in these assay systems indicates that the test compound inhibits signal transduction initiated by 4F2hc or CD98lc activation. The cell lysates can be readily assayed using a Western blot format; i.e., the host cell proteins are resolved by gel electrophoresis, transferred and probed using an antibody (e.g., an antibody labelled with a signal generating compound, such as radiolabel, fluor, enzyme, etc.). Alternatively, an ELISA format could be used in which a particular host cell protein involved in the 4F2hc and CD98lc signal transduction pathway is immobilized using an anchoring antibody specific for the target host cell protein, and the presence or absence of the immobilized host cell protein is detected using a labelled second antibody. In yet another approach, transport activity, such as that of tri-iodothyronine (T₃), thyroxine (T₄), other iodothyronines, tryptophan, or phenylalanine, can be measured as an end point for 4F2hc or CD98lc stimulated signal transduction.

In general, other cell-based screening procedures of the invention involve providing appropriate cells which express a 4F2hc-CD98lc heterodimer on the surface thereof. Such cells include cells from vertebrates, yeast, Drosophila or E. coli. In particular, one or more polynucleotides encoding a 4F2hc or CD98lc polypeptide of the present invention is employed to transfect cells to thereby express a 4F2hc-CD98lc heterodimer. The expressed 4F2hc-CD98lc heterodimer is then contacted with a test compound to observe binding, stimulation or inhibition of a functional response.

One such screening procedure involves the use of melanophores, which are transfected to express a 4F2hc-CD98lc heterodimer. Such a screening technique is described in PCT WO 92/01810, published Feb. 6, 1992. Such an assay may be employed to screen for a compound which inhibits activation of a 4F2hc-CD98lc heterodimer of the present invention by contacting the melanophore cells which encode the 4F2hc-CD98lc heterodimer with both a receptor ligand, such as T₃ or T₄, and a compound to be screened. Inhibition of the signal generated by the ligand indicates that a compound is a potential antagonist for the 4F2hc-CD98lc heterodimer, i.e., inhibits activation of the 4F2hc-CD98lc heterodimer.

The technique may also be employed for screening of compounds which activate a 4F2hc-CD98lc heterodimer of the present invention by contacting such cells with compounds to be screened and determining whether such compound generates a signal, i.e., activates the 4F2hc-CD98lc heterodimer.

Another method involves screening for compounds which are antagonists, and thus inhibit activation of a 4F2hc-CD98lc heterodimer of the present invention by determining inhibition of binding of labeled ligand, such as T₃ or T₄, to cells which have the 4F2hc-CD98lc heterodimer on the surface thereof, or cell membranes containing the 4F2hc-CD98lc heterodimer. Such a method involves transfecting an eukaryotic cell with one or more DNAs encoding a 4F2hc or CD98lc polypeptide such that the cell expresses the 4F2hc-CD98lc heterodimer on its surface (or using an eukaryotic cell that expresses the 4F2hc-CD98lc heterodimer on its surface). The cell is then contacted with a potential antagonist in the presence of a labeled form of a ligand, such as T₃ or T₄. The ligand can be labeled, e.g., by radioactivity. The amount of labeled ligand bound to the 4F2hc-CD98lc heterodimers is measured, e.g., by measuring radioactivity associated with transfected cells or membrane from these cells. If the compound binds to the 4F2hc-CD98lc heterodimer, the binding of labeled ligand to the 4F2hc-CD98lc heterodimer is inhibited as determined by a reduction of labeled ligand that binds to the 4F2hc-CD98lc heterodimers. This method is called a binding assay.

Another such screening procedure involves use of eukaryotic cells which are transfected to express a 4F2hc-CD98lc heterodimer of the present invention (or use of eukaryotic cells that express the 4F2hc-CD98lc heterodimer on their surface), and which are also transfected with a reporter gene construct that is coupled to activation of the 4F2hc-CD98lc heterodimer (for example, luciferase or beta-galactosidase behind an appropriate promoter). The cells are contacted with a test substance and a receptor agonist, such as T₃ or T₄, and the signal produced by the reporter gene is measured after a defined period of time. The signal can be measured using a luminometer, spectrophotometer, fluorimeter, or other such instrument appropriate for the specific reporter construct used. Inhibition of the signal generated by the ligand indicates that a compound is a potential antagonist for the 4F2hc-CD98lc heterodimer. Alternatively, generation of the signal generated by the ligand indicates that a compound is a potential agonist for the 4F2hc-CD98lc heterodimer.

Another such screening technique for antagonists or agonists involves introducing one or more RNAs encoding a 4F2hc or CD98lc polypeptide into Xenopus oocytes to transiently express the 4F2hc-CD98lc heterodimer. The 4F2hc-CD98lc heterodimer-expressing oocytes are then contacted with a receptor ligand, such as T₃ or T₄, and a compound to be screened. Inhibition or activation of the 4F2hc-CD98lc heterodimer is then determined by detection of a signal, such as thyroid hormone transport.

The present invention also provides a method for determining whether a ligand not known to be capable of binding to 4F2hc-CD98lc heterodimer can bind to such 4F2hc-CD98lc heterodimer which comprises contacting a eukaryotic cell which expresses a 4F2hc-CD98lc heterodimer with the ligand, such as T₃ or T₄, under conditions permitting binding of candidate ligands to a 4F2hc-CD98lc heterodimer, and detecting the presence of a candidate ligand which binds to the 4F2hc-CD98lc heterodimer thereby determining whether the ligand binds to the 4F2hc-CD98lc heterodimer. The systems hereinabove described for determining agonists and/or antagonists may also be employed for determining ligands, which bind to the 4F2hc-CD98lc heterodimer.

In addition, animal-based systems may be used to identify compounds capable of ameliorating thyroid hormone disorders. Such animal models may be used as test substrates for the identification of drugs, pharmaceuticals, therapies and interventions which may be effective in treating such disorders. For example, animal models may be exposed to a compound suspected of ameliorating thyroid hormone disorders, at a sufficient concentration and for a time sufficient to elicit an amelioration of thyroid hormone disorders in the exposed animals. The response of the animals to the exposure may be monitored by assessing the reversal of disorders associated with abnormal thyroid hormone transport such as hyper- and hypo-thyroidism. With regard to intervention, any treatments that reverse any aspect of thyroid hormone disorders should be considered as candidates for human therapeutic intervention. Dosages of test agents may be determined by deriving dose-response curves, as discussed in the sections below.

Inhibition of 4F2hc or CD98lc Expression or Activity to Ameliorate Thyroid Hormone Disorders

Any method that neutralizes TH or inhibits expression of the 4F2hc or CD98lc gene (either transcription or translation) can be used to ameliorate thyroid hormone disorders.

For example, the administration of soluble peptides, proteins, fusion proteins, or antibodies (including anti-idiotypic antibodies) that bind to and “neutralize” circulating TH can be used to decrease thyroid hormone transport. To this end, peptides corresponding to the ECD of 4F2hc or CD98lc, soluble deletion mutants of 4F2hc or CD98lc (e.g., TMD mutants), or either of these 4F2hc or CD98lc domains or mutants fused to another polypeptide (e.g., an IgFc polypeptide) can be utilized. Alternatively, anti-idiotypic antibodies or Fab fragments of antiidiotypic antibodies that mimic the 4F2hc or CD98lc ECD and neutralize TH can be utilized. Such 4F2hc or CD98lc peptides, proteins, fusion proteins, anti-idiotypic antibodies or Fabs are administered to a subject in amounts sufficient to neutralize TH and to normalize thyroid hormone transport.

For the production of antibodies, various host animals may be immunized by injection with the 4F2hc or CD98lc, a 4F2hc or CD98lc peptide, truncated 4F2hc or CD98lc polypeptides, or functional equivalents or mutants of 4F2hc or CD98lc. Such host animals may include but are not limited to rabbits, mice, and rats, to name but a few. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum. Polyclonal antibodies are heterogeneous populations of antibody molecules derived from the sera of the immunized animals.

Monoclonal antibodies, which are homogeneous populations of antibodies to a particular antigen, may be obtained by any technique that provides for the production of antibody molecules by continuous cell lines in culture. These include, but are not limited to, the hybridoma technique of Kohler and Milstein, (1975, Nature 256:495-497; and U.S. Pat. No. 4,376,110), the human B-cell hybridoma technique (Kosbor et al., 1983, Immunology Today 4:72; Cole et al., 1983, Proc Natl Acad Sci USA 80:2026-2030), and the EBV-hybridoma technique (Cole et al., 1985, Monoclonal Antibodies And Cancer Therapy, Alan R. Liss, Inc., pp. 77-96). Such antibodies may be of any immunoglobulin class including IgG, IgM, IgE, IgA, IgD and any subclass thereof. The hybridoma producing the mAb of this invention may be cultivated in vitro or in vivo. Production of high titers of mAbs in vivo makes this the presently preferred method of production.

In addition, techniques developed for the production of “chimeric antibodies” (Morrison et al., 1984, Proc Natl Acad Sci 81:6851-6855; Neuberger et al., 1984, Nature 312:604-608; Takeda et al., 1985, Nature 314:452-454) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable region derived from a murine mAb and a human immunoglobulin constant region.

Alternatively, techniques described for the production of single chain antibodies (U.S. Pat. No. 4,946,778; Bird, 1988, Science 242:423-426; Huston et al., 1988, Proc Natl Acad Sci USA 85:5879-5883; and Ward et al., 1989, Nature 334:544-546) can be adapted to produce single chain antibodies against 4F2hc or CD98lc gene products. Single chain antibodies are formed by linking the heavy and light chain fragments of the Fv region via an amino acid bridge, resulting in a single chain polypeptide.

Antibody fragments that recognize specific epitopes may be generated by known techniques. For example, such fragments include but are not limited to: the F(ab′)₂ fragments which can be produced by pepsin digestion of the antibody molecule and the Fab fragments which can be generated by reducing the disulfide bridges of the F(ab′)₂ fragments. Alternatively, Fab expression libraries may be constructed (Huse et al., 1989, Science 246:1275-1281) to allow rapid and easy identification of monoclonal Fab fragments with the desired specificity.

Antibodies to 4F2hc or CD98lc can, in turn, be utilized to generate anti-idiotype antibodies that “mimic” 4F2hc or CD98lc, using techniques well known to those skilled in the art. (See, e.g., Greenspan & Bona, 1993, FASEB J 7(5): 437-444; and Nissinoff, 1991, J Immunol 147(8): 2429-2438). For example antibodies which bind to the ECD of 4F2hc or CD98lc and competitively inhibit the binding of TH to 4F2hc or CD98lc can be used to generate anti-idiotypes that “mimic” the ECD, and, therefore, bind and neutralize TH. Such neutralizing anti-idiotypes or Fab fragments of such anti-idiotypes can be used in therapeutic regimens to neutralize TH and decrease thyroid hormone transport.

In an alternate embodiment, therapy can be designed to reduce the level of endogenous 4F2hc or CD98lc gene expression, e.g., using antisense or ribozyme approaches to inhibit or prevent translation of 4F2hc or CD98lc mRNA transcripts; triple helix approaches to inhibit transcription of the 4F2hc or CD98lc gene; or targeted homologous recombination to inactivate or “knock out” the 4F2hc or CD98lc gene or its endogenous promoter. Delivery techniques should be preferably designed to be of a systemic nature. Alternatively, the antisense, ribozyme or DNA constructs described herein could be administered directly to the site containing the target cells; e.g., muscle, brain, adipose tissue, placenta, as well as thyroid gland or liver.

Antisense approaches involve the design of oligonucleotides (either DNA or RNA) that are complementary to 4F2hc or CD98lc mRNA. The antisense oligonucleotides will bind to the complementary 4F2hc or CD98lc mRNA transcripts and prevent translation. Absolute complementarity, although preferred, is not required. A sequence “complementary” to a portion of an RNA, as referred to herein, means a sequence having sufficient complementarity to be able to hybridize with the RNA, forming a stable duplex; in the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

Oligonucleotides that are complementary to the 5′ end of the message, e.g., the 5′ untranslated sequence up to and including the AUG initiation codon, should work most efficiently at inhibiting translation. However, sequences complementary to the 3′ untranslated sequences of mRNAs have recently shown to be effective at inhibiting translation of mRNAs as well. See generally, Wagner, R., 1994, Nature 372:333-335. Thus, oligonucleotides complementary to either the 5′- or 3′-non-translated, non-coding regions of 4F2hc or CD98lc could be used in an antisense approach to inhibit translation of endogenous 4F2hc or CD98lc mRNA. Oligonucleotides complementary to the 5′ untranslated region of the mRNA should include the complement of the AUG start codon. Antisense oligonucleotides complementary to mRNA coding regions could also be used in accordance with the invention. Whether designed to hybridize to the 5′-, 3′- or coding region of 4F2hc or CD98lc mRNA, antisense nucleic acids should be at least six nucleotides in length, and are preferably oligonucleotides ranging from 6 to about 50 nucleotides in length. In specific aspects the oligonucleotide is at least 6 nucleotides, at least 17 nucleotides, at least 25 nucleotides or at least 50 nucleotides.

Regardless of the choice of target sequence, it is preferred that in vitro studies are first performed to quantitate the ability of the antisense oligonucleotide to inhibit gene expression. It is preferred that these studies utilize controls that distinguish between antisense gene inhibition and nonspecific biological effects of oligonucleotides. It is also preferred that these studies compare levels of the target RNA or protein with that of an internal control RNA or protein. Additionally, it is envisioned that results obtained using the antisense oligonucleotide are compared with those obtained using a control oligonucleotide. It is preferred that the control oligonucleotide is of approximately the same length as the test oligonucleotide and that the nucleotide sequence of the oligonucleotide differs from the antisense sequence no more than is necessary to prevent specific hybridization to the target sequence.

The oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors in vivo), or agents facilitating transport across the cell membrane (see, e.g., Letsinger et al., 1989, Proc Natl Acad Sci USA 86:6553-6556; Lemaitre et al., 1987, Proc Natl Acad Sci 84:648-652; PCT Publication No. WO88/09810, published Dec. 15, 1988) or other barriers, hybridization-triggered cleavage agents, (See, e.g., Krol et al., 1988, BioTechniques 6:958-976) or intercalating agents. (See, e.g., Zon, 1988, Pharm Res 5:539-549). To this end, the oligonucleotide may be conjugated to another molecule, e.g., a peptide, hybridization triggered cross-linking agent, transport agent, hybridization-triggered cleavage agent, etc.

The antisense oligonucleotide may comprise at least one modified base moiety which is selected from the group including but not limited to 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

The antisense oligonucleotide may also comprise at least one modified sugar moiety selected from the group including but not limited to arabinose, 2-fluoroarabinose, xylulose, and hexose.

In yet another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

Oligonucleotides of the invention may be synthesized by standard methods known in the art, e.g. by use of an automated DNA synthesizer (such as are commercially available from Biosearch, Applied Biosystems, etc.). As examples, phosphorothioate oligonucleotides may be synthesized by the method of Stein et al. (1988, Nucl Acids Res 16:3209), methylphosphonate oligonucleotides can be prepared by use of controlled pore glass polymer supports (Sarin et al., 1988, Proc Natl Acad Sci 85:7448-7451), etc.

The antisense molecules should be delivered to cells which express the 4F2hc or CD98lc in vivo, e.g., muscle, brain, adipose tissue, placenta, as well as thyroid gland or liver. A number of methods have been developed for delivering antisense DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systemically.

However, it is often difficult to achieve intracellular concentrations of the antisense sufficient to suppress translation of endogenous mRNAs. Therefore a preferred approach utilizes a recombinant DNA construct in which the antisense oligonucleotide is placed under the control of a strong pol III or pol II promoter. The use of such a construct to transfect target cells in the patient will result in the transcription of sufficient amounts of single stranded RNAs that will form complementary base pairs with the endogenous 4F2hc or CD98lc transcripts and thereby prevent translation of the 4F2hc or CD98lc mRNA. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian, preferably human cells. Such promoters can be inducible or constitutive. Such promoters include but are not limited to: the SV40 early promoter region (Bernoist and Chambon, 1981, Nature 290:304-310), the promoter contained in the 3′ long terminal repeat of Rous sarcoma virus (Yamamoto et al., 1980, Cell 22:787-797), the herpes thymidine kinase promoter (Wagner et al., 1981, Proc Natl Acad Sci 78:1441-1445), the regulatory sequences of the metallothionein gene (Brinster et al., 1982, Nature 296:39-42), etc. Any type of plasmid, cosmid, YAC or viral vector can be used to prepare the recombinant DNA construct which can be introduced directly into the tissue site; e.g., muscle, brain, adipose tissue, placenta, as well as thyroid gland or liver. Alternatively, viral vectors can be used which selectively infect the desired tissue; in which case administration may be accomplished by another route (e.g., systemically).

Ribozyme molecules designed to catalytically cleave 4F2hc or CD98lc mRNA transcripts can also be used to prevent translation of 4F2hc or CD98lc mRNA and expression of 4F2hc or CD98lc. (See, e.g., PCT International Publication WO90/11364, published Oct. 4, 1990; Sarver et al., 1990, Science 247:1222-1225). While ribozymes that cleave mRNA at site specific recognition sequences can be used to destroy 4F2hc or CD98lc mRNAs, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Haseloff and Gerlach, 1988, Nature 334:585-591. There are presumably a number of potential hammerhead ribozyme cleavage sites within the nucleotide sequence of human 4F2hc or CD98lc cDNA. Preferably the ribozyme is engineered so that the cleavage recognition site is located near the 5′ end of the 4F2hc or CD98lc mRNA; i.e., to increase efficiency and minimize intracellular accumulation of nonfunctional mRNA transcripts.

The ribozymes of the present invention also include RNA endoribonucleases (hereinafter “Cech-type ribozymes”) such as the one which occurs naturally in Tetrahymena Thermophila (known as the IVS, or L-19 IVS RNA) and which has been extensively described by Thomas Cech and collaborators (Zaug, et al., 1984, Science 224:574-578; Zaug and Cech, 1986, Science 231:470-475; Zaug, et al., 1986, Nature 324:429-433; published International patent-application No. WO 88/04300 by University Patents Inc.; Been and Cech, 1986, Cell 47:207-216). The Cech-type ribozymes have an eight base pair active site which hybridizes to a target RNA sequence whereafter cleavage of the target RNA takes place. The invention encompasses those Cech-type ribozymes which target eight base-pair active site sequences that are present in 4F2hc or CD98lc.

As in the antisense approach, the ribozymes can be composed of modified oligonucleotides (e.g. for improved stability, targeting, etc.) and should be delivered to cells which express 4F2hc or CD98lc in vivo, e.g., muscle, brain, adipose tissue, placenta, as well as thyroid gland or liver. A preferred method of delivery involves using a DNA construct “encoding” the ribozyme under the control of a strong constitutive pol III or pol II promoter, so that transfected cells will produce sufficient quantities of the ribozyme to destroy endogenous 4F2hc or CD98lc messages and inhibit translation. Because ribozymes unlike antisense molecules, are catalytic, a lower intracellular concentration is required for efficiency.

Endogenous 4F2hc or CD98lc gene expression can also be reduced by inactivating or “knocking out” the 4F2hc or CD98lc gene or its promoter using targeted homologous recombination. (E.g., see Smithies et al., 1985, Nature 317:230-234; Thomas & Capecchi, 1987, Cell 51:503-512; Thompson et al., 1989 Cell 5:313-321). For example, a mutant, non-functional 4F2hc or CD98lc (or a completely unrelated DNA sequence) flanked by DNA homologous to the endogenous 4F2hc or CD98lc gene (either the coding regions or regulatory regions of the 4F2hc or CD98lc gene) can be used, with or without a selectable marker and/or a negative selectable marker, to transfect cells that express 4F2hc or CD98lc in vivo. Insertion of the DNA construct, via targeted homologous recombination, results in inactivation of the 4F2hc or CD98lc gene. This approach is acceptable for use in humans provided the recombinant DNA constructs are directly administered or targeted to the required site in vivo using appropriate viral vectors.

Alternatively, endogenous 4F2hc or CD98lc gene expression can be reduced by targeting deoxyribonucleotide sequences complementary to the regulatory region of the 4F2hc or CD98lc gene (i.e., the 4F2hc or CD98lc promoter and/or enhancers) to form triple helical structures that prevent transcription of the 4F2hc or CD98lc gene in target cells in the body. (See generally, Helene, C. 1991, Anticancer Drug Des 6(6):569-84; Helene, C., et al., 1992, Ann NY Acad Sci 660:27-36; and Maher, L. J., 1992, Bioassays 14:807-15).

In yet another embodiment of the invention, the activity of the 4F2hc or CD98lc can be reduced using a “dominant negative” approach to normalize thyroid hormone transport. To this end, constructs that encode defective 4F2hcs or CD98lcs, can be used in gene therapy approaches to diminish the activity of 4F2hc or CD98lc in appropriate target cells. For example, nucleotide sequences that direct host cell expression of 4F2hcs or CD98lcs in which the CD or a portion of the CD is deleted or mutated can be introduced into cells (by in vivo gene therapy methods described above). Alternatively, targeted homologous recombination can be utilized to introduce such deletions or mutations into the subject's endogenous 4F2hc or CD98lc gene. The engineered cells will express non-functional 4F2hc or CD98lc (i.e., a 4F2hc or CD98lc that is capable of binding its natural ligand, but incapable of signal transduction). Such engineered cells should demonstrate a diminished response to the endogenous TH, resulting in inhibition of thyroid hormone transport.

Restoration or Increase of 4F2hc or CD98lc Expression or Activity to Ameliorate Thyroid Hormone Disorders

With respect to an increase in the level of normal 4F2hc or CD98lc gene expression and/or 4F2hc or CD98lc gene product activity, 4F2hc or CD98lc nucleic acid sequences can be utilized for the amelioration of thyroid hormone disorders by increasing TH transport into cells. Such disorders may include defects due to low TH levels in blood circulation resulting from iodine deficiency or reduced TH synthesis, or a defective 4F2hc or CD98lc. Treatment can be administered in the form of gene replacement therapy. Specifically, one or more copies of a normal 4F2hc or CD98lc gene or a portion of the 4F2hc or CD98lc gene that directs the production of a 4F2hc or CD98lc gene product exhibiting normal function, may be inserted into the appropriate cells within a patient or animal subject, using vectors which include, but are not limited to adenovirus, adeno-associated virus, retrovirus and herpes virus vectors, in addition to other particles that introduce DNA into cells, such as liposomes.

Because the 4F2hc and CD98lc gene is expressed in the muscle, brain, adipose tissue, placenta, as well as thyroid gland and liver and other tissues, such gene replacement therapy techniques should be capable of delivering 4F2hc and CD98lc gene sequences to these cell types within patients. Thus, the techniques for delivery of the 4F2hc and CD98lc gene sequences should be designed to readily cross the cell membrane, which are well known to those of skill in the art, or, alternatively, should involve direct administration of such 4F2hc or CD98lc gene sequences inside of the cells in which the 4F2hc or CD98lc gene sequences are to be expressed. Alternatively, targeted homologous recombination can be utilized to correct the defective endogenous 4F2hc or CD98lc gene in the appropriate tissue; e.g., muscle, brain, adipose tissue, placenta, as well as thyroid gland or liver.

Finally, compounds, identified in the assays described above, which stimulate or enhance the activity of normal or defective 4F2hc or CD98lc can be used to normalize the TH signaling cascade. The formulation and mode of administration will depend upon the physico-chemical properties of the compound. The administration should include known techniques that allow for a crossing of the cell membrane.

Pharmaceutical Preparations and Methods of Administration

The compounds that are determined to affect 4F2hc or CD98lc gene expression or 4F2hc or CD98lc activity can be administered to a patient at therapeutically effective doses to treat or ameliorate thyroid hormone disorders. A therapeutically effective dose refers to that amount of the compound sufficient to result in the normalization of thyroid hormone transport. The compounds of the invention are generally administered to animals, including humans.

Effective Dose

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound, which achieves a half-maximal inhibition of symptoms) as determined in cell culture. Such information can be used to more accurately determine useful doses in humans. Levels in plasma may be measured, for example, by high performance liquid chromatography.

It will be appreciated that the actual preferred amounts of active compound in a specific case will vary according to the specific compound being utilized, the particular compositions formulated, the mode of application, and the particular sites and organism being treated. Dosages for a given host can be determined using conventional considerations, e.g., by customary comparison of the differential activities of the subject compounds and of a known agent, e.g., by means of an appropriate, conventional pharmacological protocol.

Formulations and Use

The pharmacologically active compounds of this invention can be processed in accordance with conventional methods of galenic pharmacy to produce medicinal agents for administration to patients, e.g., mammals including humans.

The compounds of this invention can be employed in admixture with conventional excipients, i.e., pharmaceutically acceptable organic or inorganic carrier substances suitable for parenteral, enteral (e.g., oral) or topical application which do not deleteriously react with the active compounds. Suitable pharmaceutically acceptable carriers include but are not limited to water, salt solutions, alcohols, gum arabic, vegetable oils, benzyl alcohols, polyethylene glycols, gelatine, carbohydrates such as lactose, amylose or starch, magnesium stearate, talc, silicic acid, viscous paraffin, perfume oil, fatty acid monoglycerides and diglycerides, pentaerythritol fatty acid esters, hydroxy methylcellulose, polyvinyl pyrrolidone, etc. The pharmaceutical preparations can be sterilized and if desired mixed with auxiliary agents, e.g., lubricants, preservatives, stabilizers, wetting agents, emulsifiers, salts for influencing osmotic pressure, buffers, coloring, flavoring and/or aromatic substances and the like which do not deleteriously react with the active compounds. They can also be combined where desired with other active agents, e.g., vitamins.

For parenteral application, which includes subcutaneous, intramuscular, intraorbital, intracapsular, intraspinal, intrastemal, and intravenous injection, particularly suitable are injectable, sterile solutions, preferably oily or aqueous solutions, as well as suspensions, emulsions, or implants, including suppositories. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

For enteral application, particularly suitable are tablets, dragees, liquids, drops, suppositories, or capsules. The pharmaceutical compositions may be prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate. A syrup, elixir, or the like can be used wherein a sweetened vehicle is employed.

Sustained or directed release compositions can be formulated, e.g., liposomes or those wherein the active compound is protected with differentially degradable coatings, e.g., by microencapsulation, multiple coatings, etc. It is also possible to freeze dry the new compounds and use the lyophilizates obtained, for example, for the preparation of products for injection.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

For topical application, there are employed as non-sprayable forms, viscous to semi-solid or solid forms comprising a carrier compatible with topical application and having a dynamic viscosity preferably greater than water. Suitable formulations include but are not limited to solutions, suspensions, emulsions, creams, ointments, powders, liniments, salves, aerosols, etc., which are, if desired, sterilized or mixed with auxiliary agents, e.g., preservatives, stabilizers, wetting agents, buffers or salts for influencing osmotic pressure, etc. For topical application, also suitable are sprayable aerosol preparations wherein the active ingredient, preferably in combination with a solid or liquid inert carrier material, is packaged in a squeeze bottle or in admixture with a pressurized volatile, normally gaseous propellant, e.g., a freon.

The compositions may, if desired, be presented in a pack or dispenser device, which may contain one or more unit dosage forms containing the active ingredient. The pack may for example comprise metal or plastic foil, such as a blister pack. The pack or dispenser device may be accompanied by instructions for administration.

Thyroid Hormone Transport by 4F2hc-IU12 Heterodimers Expressed in Xenopus Oocytes

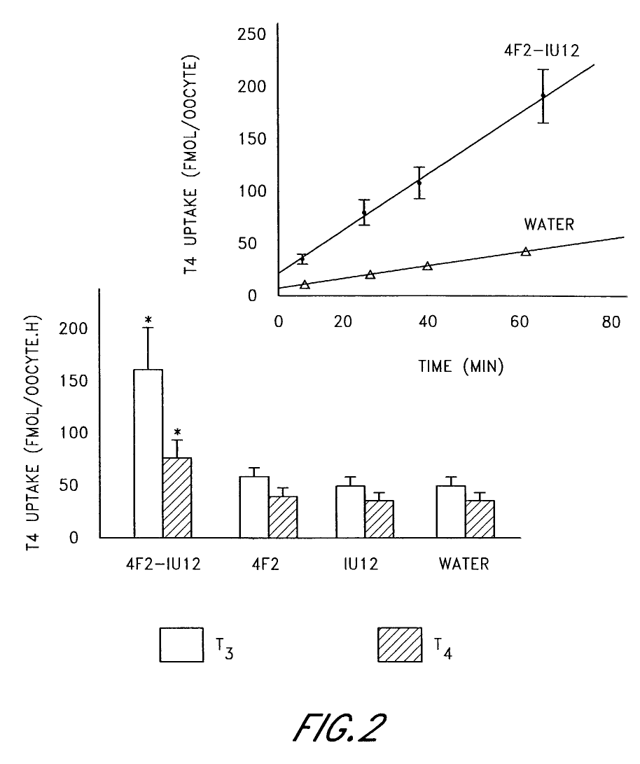

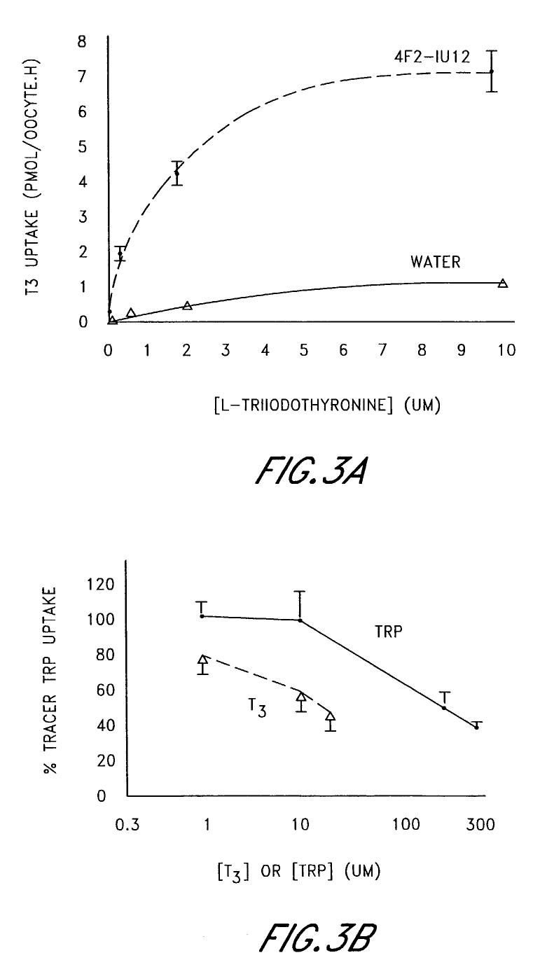

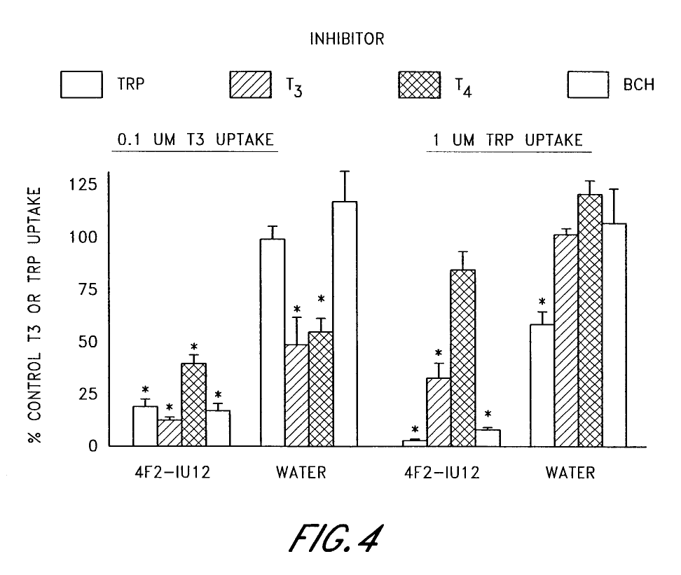

Functional expression of 4F2hc-IU12 heterodimers in oocytes after nuclear injection of cDNAs was established in preliminary experiments as a marked, 2-endoamino-bicycloheptane-2-carboxylic acid (BCH)-inhibitable induction of uptake of [³H]phenylalanine and [³H]tryptophan (averaging 120 pmol/oocyte.h for both amino acids at 50 μM) relative to uptake in water-injected cells (10 pmol/oocyte.h). Induced phenylalanine uptake was stable between 2-4 days post-injection. Uptake of both T₃ and T₄ (0.1 μM) was also significantly increased in 4F2hc-IU12 injected oocytes compared to oocytes injected with a single DNA or water (FIG. 2). The time course of [¹²⁵I]-labelled T₃ and T₄ uptake into oocytes was linear for at least 2 hours (e.g. FIG. 2); all TH studies reported below involved a 60 min uptake period using oocytes at 4 days post-injection. The average increase in uptake over control (water- or IU12-injected oocytes) was 2.1±0.5 and 2.7±0.35 times for T₄ and T₃ respectively (n=5 batches of oocytes). Induced TH uptakes were saturable and showed mutual cross-inhibition (FIGS. 3a, 4; data shown for T₃ only). Induced T₃ and T₄ uptakes measured over a concentration range of 0.05-10 μM (the latter close to the limit of iodothyronine solubility) had for T₃ an apparent Kₘ value of 1.8 μM and Vₘₐₓ of 6.4±0.3 pmol/oocyte.h (FIG. 3a) and for T₄, a Kₘ of 6.3 μM and Vₘₐₓ of 2.0±0.6 pmol/oocyte.h. Induced 0.1 μM TH uptake differed markedly from basal TH uptake measured in control (water-injected) oocytes in that it was significantly inhibited by excess BCH and tryptophan (FIG. 4, data shown for T₃ only). The 4F2hc-IU12 induced uptake of [³H]tryptophan is also inhibited by BCH (FIG. 4) and shows concentration-dependent inhibition by unlabelled T₃ and tryptophan (FIG. 3b), although inhibition by T₄ (10 μM) has not achieved statistical significance (FIG. 4). The natural iodothyronine reverse triiodothyronine (rT₃) (10 μM) inhibited induced 0.1 μM T₃ uptake by 41±4% (n=3 preparations), but the synthetic iodothyronine analogue triodothyroacetic acid (TRIAC) did not inhibit T₃ (or tryptophan) uptake in either 4F2hc-IU12- or water-injected oocytes. Substitution of TMA⁺ with 100 mM Na⁺ did not have a significant effect on TH or tryptophan uptake, nor did preincubation of oocytes with 1 nM T₃ and T₄ for 48h prior to transport measurement at 4-days post-injection.

### EXAMPLE 1

Xenopus laevis toads were purchased from the South African Xenopus Facility. Chemicals were obtained from Sigma (UK) with the exception of Collagenase A (Boehringer, UK) and Ultraspec water (Ambion, UK). Radiotracers were purchased from NEN (UK). cDNAs encoding IU12 (2.3 Kb EcoR1/Apa1 fragment from pBluescriptSK-IU12) (Liang et al. 1997, Cell Res 7: 179-193) and human 4F2hc (1.85 Kb Ecor1/BamH1 fragment from pSP65-4F2) (Teixeira et al. 1987, J Biol Chem 262: 9574-9580) were subcloned into the multiple-cloning region of pSG5 (an SV-40 driven expression plasmid).

Oocytes were isolated by collagenase treatment (Peter et al. 1996, Biochem J 318: 915-922) of ovarian tissue obtained from mature female Xenopus laevis toads (South African Xenopus Facility). Defolliculated, stage V-V1 (prophase-arrested) oocytes were selected and maintained at 18° C. in Modified Barth's Medium (MBM) containing (in mM): 88 NaCl, 1 KCL, 2.4 NaHCO₃, 0.82 MgSO₄.7H₂O, 0.66 NaNO₃, 0.75 CaCl₂x2H₂O, 5.0 HEPES, pH 7.6 with Tris base, 10 mg/liter gentamycin sulphate. For DNA injection, oocytes were transferred into individual wells of Tetraski plates pre-filled with MBM and centrifuged at 500 g for 10 minutes at 18° C., which causes migration of the nucleus to the cell surface and facilitates nuclear injection (Mertz & Gurdon 1977, PNAS 395: 288-291). The visible nucleus of each oocyte was injected with 2 ng DNA in 15 nl Ultraspec water using a pneumatic delivery system (Peter et al. 1996, Biochem J 318: 915-922). For co-injection studies, 2 ng of both DNAs (i.e. 4F2hc/IU12) were injected. Nuclei of control oocytes were injected with Ultraspec water. Oocytes were incubated in MBM at 18° C. for 4 days to allow expression of injected DNA before experimentation.

Thyroid hormone transport in oocytes was measured as influx of [¹²⁵I] labelled T₃ and T₄ tracers using a procedure described previously for amino acid uptake (Peter et al. 1996, Biochem J 318: 915-922). All experiments were carried out at 22° C. using Na⁺ free transport buffer (unless otherwise stated) containing 100 mM tetramethylammonium chloride (TMAC1), 2 mM KCl; 1 mM CaCl₂; 1 mM MgCl₂; 10 mM HEPES, pH 7.5 with Tris). Radiolabelled amino acid uptake (³H-phenylalanine or ³H-tryptophan over 30 min) was also measured (Peter et al. 1996, Biochem J 318: 915-922). BCH (5 mM) was used as a specific inhibitor of amino acid transport System L.

Data are expressed as mean values±standard error of the mean (S.E.M; n=number of observations). Experimental measurements in each batch of oocytes were made on 7-11 individual oocytes. Differences between mean values were assessed using Students unpaired t-test, with significance assigned at p<0.05.

### EXAMPLE 2

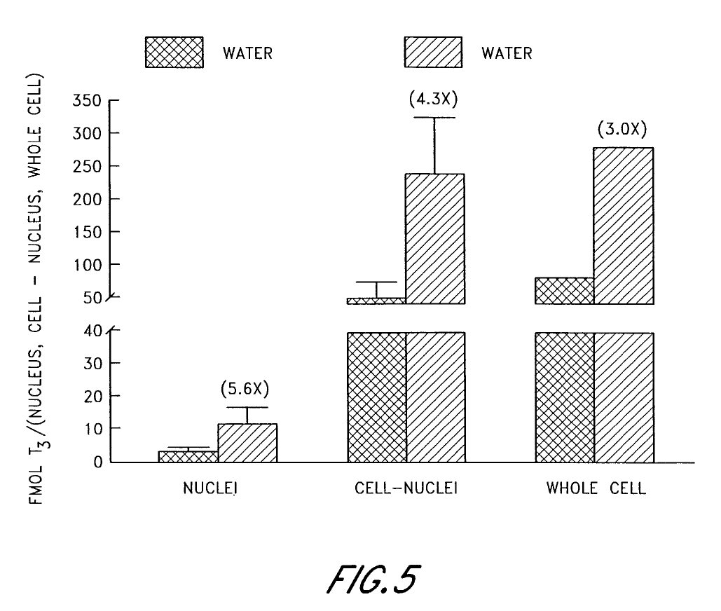

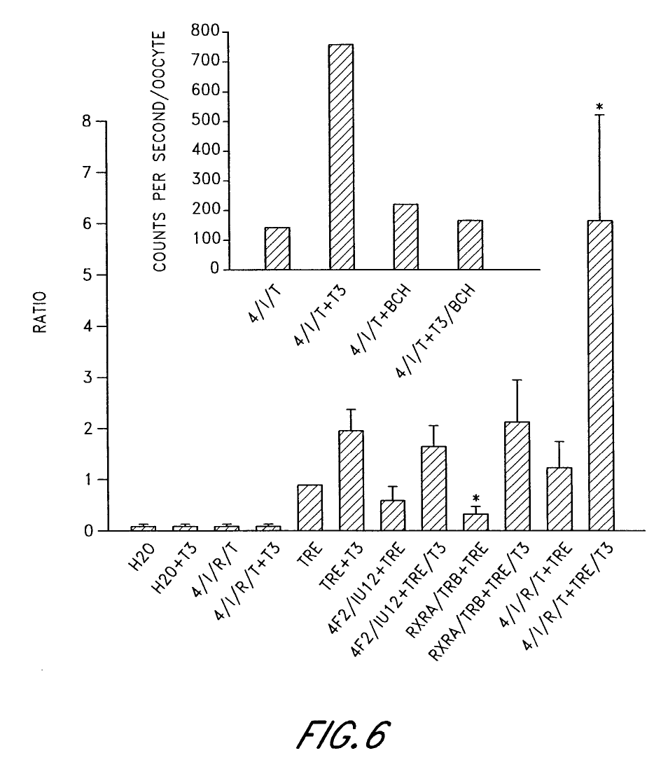

Overexpresson of the 4F2hc-IU12 heterodimer in oocytes produced a functional activation of System L transporter activity. The transporter accepts T₃ as a substrate and increased T₃ uptake through the expressed carrier results in a concomitant increase in T₃ delivery to the oocyte nucleus (FIG. 5). This showed that T₃ transport at the oocyte plasma membrane was a key step for control of T₃ transfer from extracellular medium to the cell nucleus. To confirm that increased T₃ delivery to nucleus resulted in a stimulation of gene transcription a thyroid-responsive luciferase reporter (TRE) was injected into oocyte nuclei. The construct, TREpGLB, was made by inserting a 1.6 kb fragment of the Xenopus laevis TRP A promoter regulated by T3 (Wong et al., 1995, Genes Dev 9:2696-2711) in front of the luciferase reporter gene of pGL2B plasmid (Promega). A marked increase in T₃-dependent activation of luciferase activity was observed in oocytes expressing 4F2hc-IU12, when sufficient nuclear receptors (RXRα/TRβ) were co-expressed (FIG. 6). This increase was blocked by the System L specific substrate BCH, which would compete out the increased T₃ uptake into cells overexpressing the transporter. Thus, overexpression of the System L transporter proteins leads to enhanced transport of extracellular T₃ into oocytes, resulting in increased activation of the reporter gene by thyroid hormone receptor. This result confirms the utility of regulating T₃ action through alteration of T₃ transport activity.

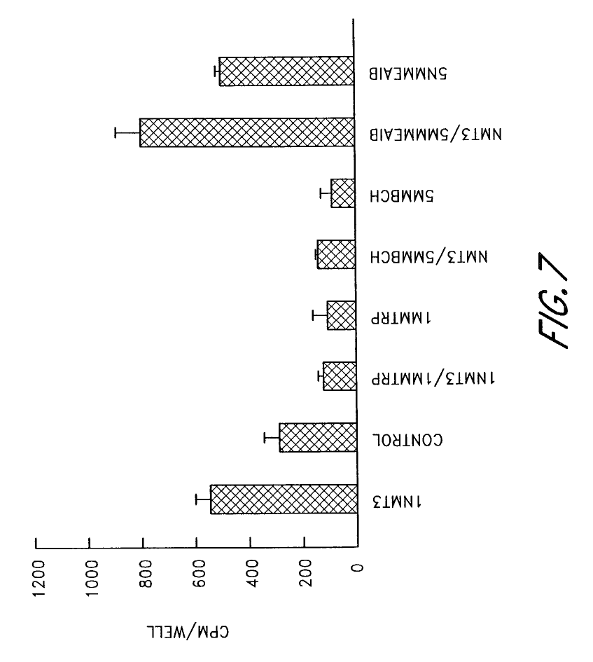

The above conclusion in frogs is also valid in mammals, as expected from the highly conserved nature of the T₃ signalling pathway during evolution. Recent studies (Ritchie, J. W. A. & Taylor, P. M., 2001, Biochem J 356: 719-725) reveal that the System L transporter also mediates uptake of T₃ into a cell-line of human origin (BeWo placental choriocarcinoma). FIG. 7 shows that tryptophan and BCH also inhibit both (i) nuclear uptake of [¹²⁵I]T₃ and (ii) T₃-dependent transcriptional activation of a luciferase reporter in BeWo cells. That is, as in frog oocyte, System L activity is important for T₃ transport and nuclear T₃ action in human cells.

Although the invention has been described with reference to embodiments and examples, it should be understood that various modifications can be made without departing from the spirit of the invention. Accordingly, the invention is limited only by the following claims. All references cited herein are hereby expressly incorporated by reference.

What is claimed is:

1. An isolated complex comprising a 4F2hc-CD98lc heterodimer and a thyroid hormone.

2. A method of ameliorating a thyroid hormone disorder comprising a step of administering a compound that modulates a 4F2hc-CD98lc heterodimer to a patient in need thereof.

3. A method of identifying a compound that can be used to modulate a thyroid hormone disorder comprising steps of:
(a) incubating a cell that expresses a 4F2hc-CD98lc heterodimer, or membrane vesicle or membrane fraction thereof, in a presence and absence of a test compound;
(b) determining an amount of thyroid hormone transport mediated by said 4F2hc-CD98lc heterodimer in the presence and absence of said test compound;
(c) selecting said test compound that alters the amount of said thyroid hormone transport; and
(d) identifying said selected compound as being a candidate compound useful for the modulation of a thyroid hormone disorder.

4. A method of identifying a compound that can be used to modulate a thyroid hormone disorder comprising steps of:
(a) incubating a cell that expresses a 4F2hc-CD98lc heterodimer, or membrane vesicle or membrane fraction thereof, in a presence and absence of a test compound;
(b) determining activity of said 4F2hc-CD98lc heterodimer in the presence and absence of said test compound;
(c) selecting said test compound that alters the activity of said heterodimer; and
(d) identifying said selected compound as being a candidate compound useful for the modulation of a thyroid hormone disorder.

5. A method of identifying a compound that can be used to modulate a thyroid hormone disorder comprising steps of:
(a) incubating a cell that expresses a 4F2hc-CD98lc heterodimer, or membrane vesicle or membrane fraction thereof, in a presence and absence of a test compound;
(b) determining whether said test compound binds to said 4F2hc-CD98lc heterodimer;
(c) selecting said test compound that binds to said 4F2hc-CD98lc heterodimer; and
(d) identifying said selected compound as being a candidate compound useful for the modulation of a thyroid hormone disorder.

6. A method of identifying a compound that can be used to modulate a thyroid hormone disorder comprising steps of:
(a) incubating a cell that expresses a 4F2hc-CD98lc heterodimer, or membrane vesicle or membrane fraction thereof, in a presence and absence of a test compound;
(b) detecting a change in expression of a 4F2hc or CD98lc gene or a change in activity of a 4F2hc or CD98lc gene product expressed by the cell;
(c) selecting a test compound that alters said expression or activity; and
(d) identifying said selected compound as being a candidate compound useful for the modulation of a thyroid hormone disorder.

7. A method of identifying an agonist or antagonist that can be used to modulate a thyroid hormone disorder comprising steps of:
(a) incubating a cell that expresses a 4F2hc-CD98lc heterodimer, and said heterodimer being associated with a second component that provides a detectable signal in response to binding of a compound to said heterodimer, in a presence and absence of a test compound;
(b) determining whether said test compound binds to said 4F2hc-CD98lc heterodimer by measuring a level of a signal generated from interaction of the test compound with said heterodimer;
(c) selecting said test compound that binds to said 4F2hc-CD98lc heterodimer; and
(d) identifying said selected compound as being a candidate agonist or antagonist useful for the modulation of a thyroid hormone disorder.

8. The method of the above claim which further comprises conducting the identification of the agonist or antagonist in a presence of labeled or unlabeled known agonist.

9. A method of identifying an agonist or antagonist that can be used to modulate a thyroid hormone disorder comprising steps of:
(a) incubating a cell that expresses a 4F2hc-CD98lc heterodimer, or membrane vesicle or membrane fraction thereof, in a presence of a known ligand, and additionally in a presence and absence of a test compound;
(b) determining whether said test compound inhibits binding of said ligand to said 4F2hc-CD98lc heterodimer by measuring an amount of said ligand bound to said heterodimer;
(c) selecting said test compound that causes reduction of binding of said ligand; and
(d) identifying said selected compound as being a candidate agonist or antagonist useful for the modulation of a thyroid hormone disorder.

10. The method of the above claim in which the ligand is labeled or unlabeled known agonist.

11. A method of making a pharmaceutical composition comprising a step of combining the compound identified by any of claims 3-10 with a pharmaceutically acceptable carrier.

12. A method of ameliorating a thyroid hormone disorder comprising a step of administering the pharmaceutical composition of claim 11 to a patient in need thereof.
